(19) United States
(12) Patent Application Publication
(10) Pub. No.: US 2016/0242646 A1
(43) Pub. Date: Aug. 25, 2016

(54) NONINVASIVE MEDICAL MONITORING DEVICE, SYSTEM AND METHOD

(71) Applicant: Padraic R. Obma, Green Bay, WI (US)

(72) Inventor: Padraic R. Obma, Green Bay, WI (US)

(21) Appl. No.: 15/079,394

(22) Filed: Mar. 24, 2016

Related U.S. Application Data
(60) Provisional application No. 62/189,431, filed on Jul. 7, 2015.
(60) Provisional application No. 62/221,229, filed on Sep. 21, 2015.

(51) Int. Cl. A61B 5/00; A61B 5/103; A61B 5/01; A61B 5/107; A61B 5/0488

(52) U.S. Cl. CPC A61B 5/0024; A61B 5/1072; A61B 5/0488; A61B 5/01; A61B 5/1032; A61B 5/6804; A61B 5/6812; A61B 2560/0475; A61B 2562/222; A61B 2562/028

(57) ABSTRACT

A variety of device constructs are contemplated in the wearable sensing devices of the present invention, each of which facilitate the monitoring of physical and physiological parameters in humans. Wearable sensing devices are incorporated into a wearable and comprise a small portable power supply; monitoring electronics mounted to or used in the wearable; a processing unit or component; and a memory unit or component to continuously or intermittently record parameter data—such data then being stored in an onboard portable memory unit and/or wirelessly transmitted to another electronic device or devices. The wearable sensing device incorporates a wireless data transmission unit or component to link to a personal computing device. The memory unit can be synchronized with the processing unit to save and then later download monitoring data for detection of any physical or physiological condition that is benign or a condition that requires mediation of some sort.

This application claims the benefit and priority of U.S. Provisional Patent Application No. 62/221,229 filed Sep. 21, 2015, and U.S. Provisional Patent Application No. 62/189,431 filed Jul. 7, 2015.

### FIELD OF THE INVENTION

This invention relates very generally to electronic data management of the type that is used to sense, measure, acquire and monitor body metrics in the medical and healthcare industries, which also includes data management in the area of athletic training and conditioning, as well as personal self-monitoring of body metrics by laypersons. As used herein, the term “body metrics” includes any physical or physiological parameter related to the human body that can be measured in an objective fashion (the terms “body metrics,” “physical or physiological parameters” and “parameters” are used interchangeably throughout this written disclosure).

The present invention also relates very generally to clothing, portions of clothing, such as sleeves and leggings, body wraps, including braces and supports, and body-attachable patches that are identified in this application as “wearables,” such wearables incorporating “wearable sensing devices,” such wearable sensing devices incorporating electronic “sensors” or “sensing elements” (the terms “sensors” and “sensing elements” are used interchangeably throughout this written disclosure) that are constructed and used to acquire data relating to body metrics. The electronic sensing elements, and the methods for using them, measure a wide variety of body metrics, such sensing elements and methods being multifunctional where feasible and further being noninvasive, i.e., not subcutaneous, in application. In this last regard, it is to be understood that the human body is sheathed and protected by an outer layer of skin, which is the integumentary system comprised of an epidermis, dermis and hypodermis. In the context of the present invention, certain physical and physiological parameters can be derived from monitoring the user's skin, including skin disposed in the vicinity of the user's joints, or by monitoring muscles that are disposed at the hypodermis or deeper, i.e., subcutaneously. In the present invention, the sensing elements are intended to be completely “non-invasive” which shall mean that the sensing is accomplished without penetrating the user's skin.

The terms “sensor” and “sensing elements” shall further mean any general or special purpose mechanism or electromechanical sensor of the type that can measure any number of body metrics. Such sensing elements include, but are not limited to, sensors for determining spatial relationships, positions and changes in position in ten or nine axes, accelerometers, magnetometers, gyroscopes, barometers, range of motion (“ROM”) sensing elements, and global positioning system (“GPS”) sensing elements, among others. There are, however, sensing devices that sense other numbers of axes, such as three axis and six axis sensors, in addition to the nine axes and ten axes sensors mentioned above, and such sensors can be combined as desired or required. For example, a ten axis sensor may be used with a six axis sensor, or a nine axis sensor may be used with a three axis sensor, and so forth.

More particularly, the present invention relates to the incorporation of one or more medical monitoring functionalities into wearable patches, wraps, clothing and clothing portions such that a wide variety of body metrics can be noninvasively monitored and then mediated as may be desired or required. As used herein, the term “monitor” shall mean the detection and assessment of a medical or physical condition or one or several body metrics, as assessed instantaneously, in real time or over an extended period of time as may be desired or required. The term “mediate” shall mean the detection and reactionary response to a medical or physical condition that has been, or is capable of, being monitored. The monitoring and mediating functionalities and methodologies presented herein may be used for assessing “digital health.”

At the core of the present invention, is the concept of utilizing “dueling sensors.” As used herein, the term “dueling sensors” is intended to define at least two sensors whereby the sensors can monitor, detect and compare a body metric via one sensor with the same metric “delta” (or an incremental change in that body metric from one sensor in relation to the other) being detected by at least one other sensor. In this way, the pair of dueling sensors can determine, in a relative way, how a sensed parameter differs between the sensors. On detection of a delta in that parameter, the parameters are inputted into a computer processing device and an algorithm is used to provide an “end-state,” which may or may not be a desired outcome but which will provide the user or healthcare provider with important information and feedback in the form of body metric data.

As applied to a wearable such as a knee support or brace, for example, a pair of dueling sensors, or even just one of them, could detect certain “activity tracking” parameters such as distance traveled, calories burned, number of flights of stairs climbed, and the like. However, the same pair of dueling sensors, when comparing the position of a first sensor in relation to a second sensor (such as where the first sensor is positioned below a knee joint and the second sensor is positioned above the same knee joint), could also detect knee joint parameters such as range of knee motion (“bend”), knee alignment (“twist” or “flex”), time spent with the knee flexed more than 90°, and similar comparative parameters. Indeed, the present invention is particularly drawn to the use of dueling sensors wherein the sensors are placed to one side of a human joint and to the other side of the human joint, the joint being disposed between the dueling sensors.

Further, and regardless of the measured parameter, a centralized microprocessor can be used to apply the necessary algorithms to those parameters and provide feedback to the user and/or to a healthcare provider as desired or required. That is, the present invention is generally related to the application and use of multiple computer processing algorithms, which algorithms comprise finite sets of rules or detailed computer instructions and each varying in complexity and each being designed to perform specific computing tasks via the centralized microprocessor. However, a processor can also be built into each of the wearable sensing devices with one such processor serving as a “hub” for processing the objective metric data relative to specific physiologic parameters detected by a plurality of other like-configured sensors that are used in concert with the wearable sensing devices. The methodology of the device and system of the present invention essentially provides a structural view of performance and relays data in three dimensions (“3-D”) through specially designed algorithms to provide active coaching and exercise tips. This is accomplished via a mobile application, or “app.”

### BACKGROUND OF THE INVENTION

Electronic devices, systems and methods are well known in the sports, athletic performance and medical arts for monitoring objective physical and physiological parameters in humans. Such devices, systems and methods typically comprise a sensing device, a processing component that is electronically connected (via hard wiring or via wireless transmission) to the sensing device and a visual display device, such as a monitor or other screen display. Other devices, systems and methods of this type can also comprise more than one sensing devices, at least one processing component that is electronically connected to all of such sensing devices, communication links for sending electronic signals (again, via hard wiring or via wireless transmission) from the multiple sensing devices via the processing component to the viewable monitor of a visual display device or to a local or remote medical information network. Transmitting data from such a sensing device is typically accomplished via a communications link, such as a transmitter that enables wireless biotelemetry and ambulatory wireless biotelemetry. Some devices of either type are provided bedside, in a setting where the patient is non-ambulatory, whereas others can be provided where the patient is fully ambulatory or where the patient is not a patient at all, but is an athlete in training, for example.

One of the most familiar monitoring devices of this type is the common inflatable arm or wrist cuff that can be used to detect and measure a patient's blood pressure and pulse. Another well-known monitoring device is the infrared finger clip that is used at the distal end of a digit to detect and measure the saturated percentage of oxygen in the blood of a patient using infrared technology. Yet another well-known monitoring device is the electromyograph (“EMG”) which is used to sense and measure the electrical activity of human muscles. EMG technology can be conducted subcutaneously, which is invasive, or via surface EMG, which is non-invasive and where skin surface electrodes assess muscle activity from the skin surface immediately above or atop the subcutaneous muscle. Again, such sensing and monitoring devices may be portable, but often are not. Other sensing devices of the type that are intended to be portable include thoracic transducer belts for monitoring respiratory rates and Holter monitors that record the electrical activity of the heart over a period of time using electrodes that are placed on a patient's body, typically over bones to minimize artifacts from muscular activity. The electrodes are sensors that are used to detect electrical changes on the patient's skin that arise from the heart muscle depolarizing during each heartbeat, which is also known as electrocardiogram (“ECG”) detection and measurement.

It is known in the art to provide wearable technology for the purpose of capturing motion positions, which is typically associated with sports and physical training activities to maximize performance, reinforce suitable muscle memory, prevent injuries and provide some limited data analytics. However, in the overwhelming majority of the currently-available types of wearable technology, basic single-purpose sensors are configured to sense a single physical parameter (e.g., sensor location or position) or slightly more complex multi-purpose sensors are configured to sense multiple parameters (e.g., sensor location of several points along a human limb, such as at a wrist, an elbow and a shoulder, or coupled with means for detecting sensor acceleration or deceleration). In the experience of this inventor, however, such wearable technology of current manufacture tends to be cumbersome and somewhat limited in scope of use. That said, the miniaturization of electronics to unprecedented levels and the ongoing development and implementation of microelectromechanical systems (“MEMS”) in a broad spectrum of applications, sensors and processors can, and should, be incorporated into a wide variety of human wearables and wearable sensing devices as well. The combined use of such electronics with wearables is not only possible, but is also desirable in that athletic and patient monitoring can be done non-invasively and with minimal interference to an athlete's episodic training performance or, in the case of medical application, with a patient's normal day-to-day activities.

In the view of this inventor, what is needed in the medical arts, as well as in the athletic training and performance arts, are wearables and wearable sensing devices that can be used to monitor any number of physical and physiological human parameters in a new and unique way. For example, one such wearable and wearable sensing device could be used to monitor leg swelling or sense a change in the circumference of a patient's leg, either of which could be an indicator of one of several deleterious post-operative complications. Circumference measurement could also be used to determine if a muscle, or muscle group, is in recovery (demonstrated by an increased circumference) or is in atrophy (indicated by a decreased circumference). Another such wearable could detect skin temperature—an increase in temperature similarly being an indicator of a localized or systemic infection. Irrespective of whether such wearables and wearable sensing devices serve as diagnostic tools and monitors of physical and physiological parameters in medical patients or as feedback devices for athletes, the electronics, or at least a portion of them, need to be incorporated directly into the wearable sensing devices which are, in turn, incorporated directly into the wearable. Some sensors can be disposed to the outside of the wearable whereas other sensors require that they be disposed to the inside of the wearable, adjacent the skin of the patient or athlete in order to achieve the functional parameter detection that is desired or required.

In the view of this inventor, there is also a need in the medical and athletic arts to provide wearables that can be variably interfaced with spatial or positional sensing means to detect metric deltas to very small but precise degrees. As alluded to above, current technology places a positional sensing device on joints or limbs for purposes of tracking movement or relational movement, shifting and positioning of that joint or limb. Such sensing devices provide feedback for a whole host of purposes such as perfecting a desired tennis overhand tennis serve, correcting body mechanics to achieve a better golf swing or analyzing body posture to enhance accuracy on a pistol shooting range.

In accordance with the present invention, however, such a wearable could be improved by providing a wearable sensing device with a sensing element or sensor to monitor the relative position of a patient's joint by assessing specific deltas above or below—or, more accurately, the relative position of points to either side of a patient's joint. By definition, a “joint” is the site of the junction of two or more bones of the body—its primary function being to provide motion and flexibility to the frame of the body. Further, most joints allow considerable motion, the most common type being “synovial joints” which have a complex internal structure which is composed of the ends of bones, ligaments, cartilage, the articular capsule, the synovial membrane and sometimes serous sacs, or bursa. For example, the knee joint is a compound joint, which is a type of synovial joint, between the femur, the patella and the tibia. The elbow joint is the synovial joint between the humerus, the ulna and the radius.

In the view of this inventor, there is a need to more accurately assess very specific changes in joint position and to assess such changes more precisely. This would preferably be done via a ten axis motion sensor that is capable of detecting rotation rates or angular velocities of the sensor, or a multiplicity of sensors (when attached to the patient), about the x, y and z axes of a Cartesian coordinate system (via a gyroscopic component or other positional relationship component) as well as axial acceleration (via an accelerometer component) and ambient magnetism (via a magnetometer component which is used to establish initial sensor calibration), all measured within the same coordinate system. However, it is also to be understood that motion sensors that sense other numbers of axes, such as three axis, six axis and nine axis sensors can be used in the present invention and that such sensors can be used in combination with other sensors, also selected from the group of three axis, six axis, nine axis and ten axis sensors. That is, a ten axis sensor may be used with a six axis sensor, or a nine axis sensor may be used with a three axis sensor, and so on, all to the same end.

Irrespective of the number of axes used in each sensor, all measuring is done via the sampling of objective measurement data detected from sensors within the wearable sensing devices in accordance with a pre-programmed scheme as determined by applied algorithms residing within a microprocessor. Further, optimal use of such a wearable sensing device would be its ability to detect one or more physical or physiological parameters and then wirelessly transmitting those metrics, in real time and via biotelemetry, to a remote server and memory unit that would then electronically store the transmitted data in a database. However, both types of wearables are the subject of the present invention—wearable and wearable sensing devices having sensors that receive and store data via a transitory memory and wearables and wearable sensing devices having sensors that receive and transmit data to a non-transitory memory.

As alluded to above, the scale of the electronics that are contemplated for use in the wearable sensing devices and methodology of the present invention must be relatively small and unobtrusive—almost to the point of being undetectable, such as by using a MEMS platform and integrated circuit configurations. If possible, the electronics (including the sensors, processors, memory, input/output (or “I/O”) components and power supply, together with hard-wired electrical connections between components) would be built directly into the wearable sensing device. As alluded to previously, some sensing elements used within the wearable could be adhered to the outside surface of the wearable whereas others could be adhered to the inside surface, and adjacent the user's skin. Alternatively, wearable sensing devices would most desirably be placed into positions by virtue of a “band-aid”-type application or patch, where the wearable sensing device is adhered directly to the users skin via medical adhesive.

Lastly, it would be desirable that the sensors used in the wearable sensing device be capable of carrying an on-board electronic power supply, such as a coin-type disposable battery or a rechargeable battery. This would allow for repeated use of the sensor upon depletion of the electric charge carried by the battery. It is also desirable, in some applications, to devise such a sensing device where the battery possesses sufficient life for useful application without the need to charge the battery. In short, the sensing device would be a replaceable consumable.

### SUMMARY OF THE INVENTION

In view of the foregoing, a wide variety of body metric sensing or wearable sensing device constructs are contemplated, devised and presented, all of which facilitate the monitoring of physical and physiological parameters in human subjects, be they medical patients, professional athletes, casual athletes or laypersons. Such wearable sensing devices are incorporated into systems and used in methods drawn to the use of such devices and systems.

The simplest construct in accordance with the present invention would be to use one wearable sensing device secured above a joint and another wearable sensing device secured below the same joint. Each wearable sensing device would necessarily require the incorporation of, or integral combination within a housing, the following: (a) one or more sensing elements; (b) a local memory; (c) a local microprocessor; (d) an on-board power supply; and (e) a local low energy wireless transceiver that would provide a wireless personal area network for use of the wearable sensing device with a smartphone. The low energy functionality is intended to provide considerably reduced power consumption and cost while also providing a sufficient wireless communication range. The smartphone would provide a user interface and an interactive screen display for the user, together with additional processing capabilities and memory. Irrespective of the mode used to secure each sensing unit, each positioned as mentioned above, the wearable sensing devices would “calibrate” from an initial position of one sensing device relative to the initial position of the other sensing device. In this last regard, at least one of the wearable sensing devices would serve as a compass for the sensing devices via a magnetometer. Following calibration, each wearable sensing device would be wirelessly queued via the smartphone to commence the gathering of body metrics. The data compiled by acquisition of the body metrics is stored within a local memory and/or be transmitted, via continuous real-time feed or via a “data dump” at a later time, to the smartphone via the low energy wireless transceivers. In this construct, each wearable sensing device could incorporate means for recharging the on-board power supply via inductive charging or other energy transfer means for re-use of the wearable sensing device.

Another construct would be to incorporate the above-referenced wearable sensing devices into a sleeve for a joint. In this construct, the sleeve would incorporate two pockets—each for housing a wearable sensing device. In this construct, it would be desirable for the housings of the wearable sensing devices to be configured to easily slide into the pockets and to be snuggly retained in them so as to prevent any movement of the wearable sensing device within the pocket. That is, any “slop” or lateral movement of the wearable sensing device within the pocket could result in the acquisition of false metrics or measurements relative to the joint.

Another construct would utilize one of the wearable sensing devices as identified above and a second wearable sensing device wherein the second wearable sensing device would be configured without a low energy wireless transceiver within it. Instead, the wearable sensing devices in this construct would be “hard wired” to one another. The metrics would be monitored using the dueling sensor concept, but only one wearable sensing device would transmit data via a low energy wireless transceiver. This alternative construct would reduce cost of the second wearable sensing device by eliminating the local low energy wireless transceiver within it. This alternative configuration would, however, function as described above in every other respect.

Another construct would be to provide two wearable sensing devices of the type that are not configured to have a local low energy wireless transceiver within them. Instead, both of the wearable sensing devices in this construct would be “hard wired” to another centralized processing unit. The centralized processing unit would be provided with the necessary functionality of the local low energy wireless transceiver, as described above. In all other respects, the configuration would also function as described above. Other sensing devices or elements could likewise be hard wired to the centralized processing unit to provide for the measurement of other body metrics.

Yet another construct would be to provide a sleeve for a leg or knee joint whereby at least one sensing element used within the wearable sensing device is incorporated into the sleeve such that swelling and concomitant circumference changes in the limb are monitored and detected. Such a wearable sensing device would necessarily require the incorporation of (a) a small on-board portable power supply; (b) limb circumference monitoring electronics in the form of longitudinally-extendable wires woven into the fabric of the wearable, or other design expediencies that would allow for measurement of such limb circumference, including a length of non-extendable material, the ends of which would be secured within a sensor having a stretch sensor, for example; (c) a processing unit or computing component; and (d) a memory unit or component to continuously or intermittently record limb circumference data—such data then being stored in an onboard portable memory unit and/or wirelessly transmitted to another electronic device or devices. In the case of the latter, the wearable would necessarily also incorporate (e) a wireless data transmission or biotelemetry unit, i.e., a transmitter or a transceiver, to enable telehealth or telemedical functionalities.

This construct could also incorporate pre-programmed instructions that are performed by the processing unit in implementing algorithmic steps to process the body metric measurements and provide the patient or the healthcare provider with real-time or time-delayed information concerning the patient's well-being in view of the measurements made. The algorithmic steps in accordance with the present invention utilize applied “quaternion” matrix mathematics, which are used to determine a rotation angle and the vectored direction of a rotation. Quaternions are used typically for calculations involving three-dimensional rotation, and describing spatial rotations in particular, while being more compact and quicker to compute than representations by other vector matrices. Lastly, the memory unit of this construct could also be synchronized with the processing unit to save and then later download monitoring data for short term detection or long term assessment of any physical or physiological condition that is benign or that requires mediation of some sort.

In another construct, a leg or knee wearable in accordance with the present invention could incorporate sensors to monitor parameters of temperature, pressure and joint angles in the knee. In yet another construct, a foot or ankle wearable could incorporate the same sensors for assessing the foot health of a diabetic patient. The monitored parameters could be used to inform the patient and his or her healthcare providers of any changes that would require mediation of a potentially problematic medical condition.

In yet another construct, a combination of sensors could be used to monitor multiple joints within an upper or lower limb or extremity. For example, a sensor could by placed above the shoulder (i.e., humeral) joint, at the middle of the upper arm, at the middle of the forearm and on the hand (i.e., four sensors in all) to measure three joints—the shoulder joint, the elbow joint and the wrist—in much the same relative fashion as described above.

In still another construct, cervical spine motion could be monitored via a sensor placed on the upper-most portion of the neck and another at the upper-most portion of the thoracic spine. Likewise, lower back, or lumbar spine, range of movement could be monitored using multiple sensors placed in strategic locations as well.

The construct of the present invention could also be incorporated into “orthopedic wearables” such as knee braces or sleeves, elbow braces or sleeves, ankle braces or sleeves, compression stockings, arm slings and the like. Sensors of the type described and claimed herein can be used in conjunction with orthopedic wearables such that data can be obtained for the purpose of decreasing complications and improving surgical or other treatment outcomes. Constructs of this nature can include sensors that are incorporated directly into the orthopedic wearable or, alternatively, sensors that are simply applied to the skin or included within a sleeve or other holder which can then be used with a conventional orthopedic brace that would then overlay the sensors.

As alluded to at the outset, the present invention also significantly relates to the novel concept of utilizing “dueling sensors.” The concept of “dueling sensors” is intended to define at least two sensors whereby the sensors can monitor, detect and compare a parameter of one sensor with the same parameter detected by another sensor. In this way, the pair of dueling sensors can determine how a sensed parameter differs between the sensors relative to their placement on a human body. As applied to a knee support or brace, a pair of dueling sensors could, for example, detect activity tracking parameters such as distance traveled, calories burned, number of flights of stairs climbed and other similar location-tracking parameters. However, the same pair of dueling sensors could also detect range of knee motion, knee alignment, time spent with the knee flexed more than 90° and virtually any other like-monitored joint parameters, as previously described.

The functionality of the present invention provides a novel solution that is itself inextricably tied to, and is necessarily rooted in, computer technology. The sensors of the wearable sensing devices secure specific data manipulations, data transformations and data transmissions that are performed by a local and integral microprocessor or by a remote microprocessor, thereby implementing the algorithmic steps as are pre-programmed within either. In alternative constructs and embodiments, other configurations for monitoring physical and physiological parameters, and for providing correlating data and feedback concerning those parameters, in real-time or time-delayed assessment format, are disclosed in the detailed description that follows, all of which are included within the scope of the present invention and the present invention not being limited to the specific embodiments disclosed.

The foregoing and other features of the present invention will be apparent from the detailed description that follows.

### BRIEF DESCRIPTION OF THE DRAWINGS

FIG. 1 is a front elevation view of a portion of a patient's leg (specifically, the patient's lower thigh, knee and upper calf) encircled by one embodiment of a wearable of the type that is constructed in accordance with the present invention.

FIG. 2 is a left side elevation view of the leg and knee wearable shown in FIG. 1.

FIG. 3 is a front elevation view of a portion of a patient's leg (again, the patient's lower thigh, knee and upper calf) encircled by an alternative embodiment of a wearable of the type that is constructed in accordance with the present invention.

FIG. 4 is a left side elevation view of the leg and knee wearable shown in FIG. 3.

FIG. 5 is a front elevation view of a portion of a patient's leg (again, the patient's lower thigh, knee and upper calf) encircled by yet another alternative embodiment of a wearable of the type that is constructed in accordance with the present invention.

FIG. 6 is a left side elevation view of the leg and knee wearable shown in FIG. 5.

FIG. 6A is an enlarged left side elevation view of the wearable taken along line 6A-6A of FIG. 6 and showing the wearable sensing device inserted into a pocket that is formed within the wearable.

FIG. 7 is a front elevation view of a portion of a patient's leg wherein the patient's lower thigh and upper calf (immediately above and below the knee, respectively) each has attached to it still another alternative embodiment of a strip-like wearable of the type that is constructed in accordance with the present invention.

FIG. 8 is a left side elevation view of the wearables shown in FIG. 7.

FIG. 9 is an isometric view of a pair of wearable sensing devices, each having one or more sensing elements or sensors (i.e., “dueling sensors” as between each of the sensing devices) embedded within each of the wearable sensing devices in accordance with the present invention wherein the devices are shown in relation to the x, y and z axes of a Cartesian coordinate system.

FIG. 10 is a front elevation view of a pair of sensors shown in FIG. 9 wherein one of the sensing devices is rotated about the z axis in relation to the other sensing device, as would be the case where the sensing devices are placed to either side of a joint, which demonstrates a “bend” relationship relative to the joint.

FIGS. 11A, 11B and 11C are left side elevation views of the pair of sensors shown in FIG. 10 wherein one of the sensing devices is rotated about the y axis in relation to the other sensing device, as would be the case where the sensing devices are placed in vertical positions to either side of a joint and the joint is showing rotation or “twist.”

FIGS. 12A, 12B and 12C are top plan views of the sensing devices shown in FIGS. 11A, 11B and 11C, respectively.

FIGS. 13A, 13B and 13C are left side elevation views of the pair of sensors shown in FIG. 10 wherein one sensing device is rotated about the x axis in relation to the other sensing device, as would be the case where the sensing devices are placed in vertical positions to either side of a joint and the joint having exaggerated valgus and varus joint alignments, or “flex.”

FIGS. 14A, 14B and 14C are views that correlate to those shown in FIGS. 13A, 13B and 13C when viewed from the z-axis.

FIG. 15 is a schematic representation of a first microelectromechanical system (“MEMS”) configured in accordance with the present invention.

FIG. 16 is a schematic representation of a second MEMS configuration in accordance with the present invention.

FIG. 17 is a schematic representation of the electronics that would be used in one system embodiment of the present invention, that embodiment being shown in FIG. 1 and the components being hard wired to a processing component having wireless connectivity to a personal computing device.

FIG. 18 is a schematic representation of the electronics that would be used in an alternative system embodiment of the present invention, that embodiment being shown in FIG. 2.

FIG. 19 is a schematic representation of the electronics that would be used in yet another alternative system embodiment of the present invention, that embodiment being shown in FIG. 3.

FIG. 20 is a flow chart illustrating the steps taken to process data captured via a sensor or a plurality of sensors.

FIGS. 21 through 34 illustrate representative screen displays shown on a user's personal computing device in accordance with the present invention.

### DETAILED DESCRIPTION

The noninvasive medical monitoring device, system and method that is configured in accordance with the present invention necessarily comprises a pair of wearable sensing devices, each sensing device comprising at least one sensor or sensing element. There can be, and preferably are, more than one sensor used in any of the preferred embodiments stated herein, with two ten axis sensors being desired, although other sensor combinations could be used. That is, sensing devices that sense other numbers of axes, such as three axis, six axis and nine axis sensors, can be used in the present invention and such sensors can be combined as desired or required. For example, a ten axis sensor may be used with a six axis sensor, or a nine axis sensor may be used with a three axis sensor, and so on.

In their most basic constructs, the sensors comprise devices for detecting a wide variety of objectively different physical parameters, such as the amount of light as detected by a light sensor; heat and cold as detected by temperature sensors; movement as detected by motion sensors, applied force as detected by pressure sensors; the presence or absence of certain harmful agents as detected by chemical sensors; electric field sensors; magnetic field sensors; displacement sensors; and acceleration sensors. Sensors of this nature are used for detecting absolute parameter values and, in more sophisticated models, used for detecting parameter deltas, or changes. Most importantly, however, is the fact that the “resolution” of a sensor is the smallest change that the sensor can detect in the relevant “quantity” that it is measuring, i.e. temperature measured in tenths of degrees Fahrenheit or Celsius; motion and displacement measured in inches, fractions of inches, millimeters, micrometers and smaller displacement distances; pressure in terms of force per unit area; and so on. In short, while such sensors typically measure “absolutes,” coupled with suitable software, algorithmic steps and memory, changes in parameters can be detected and monitored as well. These changes, or deltas, are an essential element of the present invention.

It should also be noted that each type of sensor mentioned above may have alternative terms that they are known by in the relevant arts—such as, for a pressure sensor, a pressure transducer or a piezometer and, for a force sensor, a load cell and so on—the point being that many types of sensors are available for sensing many objectively different physical parameters—any one or more of them being capable of incorporation into the monitoring device and methodology of the present invention.

In the medical arts, the types of sensors used have naturally and necessarily expanded into internal metabolic indicators, such as oxygen saturation levels and the like. In a specific medical application, such a sensor may be used to monitor and detect swelling and/or circumference changes in a limb as compared to a “baseline,” which would be a specific value or number of values (as in value ranges having an upper limit and a lower limit) that can serve as a comparison or control for that particular physical parameter.

Significantly, the wearable sensing devices and method in accordance with the present invention utilize a gyrometer for measuring limb joint rotation and an accelerometer to measure speed and directional changes in limbs or limb parts. More specifically, certain applied algorithmic steps are used to accomplish these measurements. The algorithmic steps in accordance with the present invention utilize applied “quaternion” matrix mathematics, which are used to determine a rotation angle and the vectored direction of a rotation. Quaternions are used in particular for calculations involving three-dimensional rotation, and describing spatial rotations in particular, and are more compact and quicker to compute than are representations by other vector matrices. As applied to the present invention, the accelerometer provides the amplitude of force in terms of “G-forces” (with “G” from the word “gravitational”), G-force being a measurement of the type of acceleration that causes weight. Viewed another way, physical parameters that are analyzed according to this aspect of the present disclosure include a “mass” in “motion”—the “mass” being a limb or limb part—as G-force can also be described as a “weight per unit mass”. The term “motion” can encompass rotation, reciprocation, oscillation, gyration, combinations thereof, or any other continuous, alternating, periodic, repetitive and/or intermittent change to the location or arrangement of the limb or limb part.

In the quaternion math matrix concept mentioned above, a magnetometer is also necessarily incorporated to measure directional orientation of the patient, the patient's limb or a limb part—the magnetometer providing a fixed point for the sensor in 3-dimensional space. This is an important addition as the magnetometer provides a fixed point in space that can be used to determine the spatial relationship between any two sensors. In the wearable sensing devices of the present invention, the magnetometer in a first wearable sensing device provides a point for initial calibration, or the point of start for positional changes to be detected, which essentially serves as a compass in the dueling sensor concept disclosed and claimed herein. In short, the magnetometer gives the gyrometer and accelerometer combination fixed points to calibrate from. Without the magnetometer, the only parameter that can be established is the distance between any two of the sensors, which is dynamically variable in almost all instances—one example being where one sensor is located above a joint and one is located below the joint. Upon continuous flexing of the joint, the distance between the two sensors is likewise continuously changing as is the relative rotation of the one sensor based on its orientation in relation to the other. This concept will be apparent later in this detailed description.

As an adjunct or alternative sensing element relative to the magnetometer could be a global positioning system (or “GPS”). GPS is a desirable functionality due to the fact that the magnetometer may not precisely detect and respond to an ambient magnetic field. That is, the magnetometer, more so than other MEMS-type sensing elements, is subject to undesirable magnetic fields of the type that can be generated by any number of electrical or electromechanical devices. Such fields can potentially interfere with conventional magnetometers, thereby making GPS functionality a desirable alternative in wearables that are made in accordance with the present invention. Multiple sensors could be used, and the GPS technology can be built into each sensor. Further, it would be possible for the sensors to “synch” with a personal computing device, which computing device could provide the sensor or sensing element with baseline GPS coordinates that would originate from the personal computing device. Movement of the sensor would be relative to the change in the GPS reading of the personal computing device, assuming that the personal computing device is in close proximity to the sensing element or the wearable sensing device.

Another sensor that can be used in the wearable sensing device and method of the present invention is a precision barometer, for measuring atmospheric pressure changes which can correlate to changes in elevation—even relatively small changes in elevation on the order of several inches.

The device and method in accordance with the present invention could also specifically comprise a sensor to monitor and detect skin temperature as compared to a baseline. While thermometers are well known in the medical arts, the temperature sensor of the present invention is miniaturized and adapted to be surface-mounted to the interior of a wearable, immediately on top of the skin.

Another device and method in accordance with the present invention would be a sensor to monitor and detect skin color or changes in skin color as compared to a baseline. The skin color sensor would comprise a light-emission component such as a light emitting diode (“LED”) coupled with light receiving component for the detection of the skin surface color based on skin reflectivity.

Yet another device and method in accordance with the present invention would comprise flexion, extension and positional sensors for measuring joint parameters such as joint range of motion (in degrees and minutes) as previously discussed. A limb strength sensor for measuring strength of a limb under flexion or extension could also be used in a wearable in accordance with the present invention.

Another wearable sensing device and method in accordance with the present invention could utilize electromyography (“EMG”), which is another type of electro-diagnostic technology. EMG is a technique for measuring the electrical potential generated by muscle cells and detecting the activation level or electrical potential of such cells. The activation level or electrical potential signals of such muscle cells can be used to detect medical conditions, including the biomechanics of patient movement. Detection of these levels or signals comprises the use of a muscle measurement device such as an electromyography which can produce a record over time, known as a electromyogram. This EMG technology can likewise be built into a wearable in accordance with the present invention.

Others sensing element constructs in accordance with the present invention could include a pulse rate monitor (sensing heart rate); a blood oxygenation monitor (pulse and oxygen saturation levels as compared to a baseline); a blood pressure monitor; a hemoglobin and/or hematocrit monitor; and other types of metabolic sensors.

Each sensor of the type mentioned above is incorporated into the wearable sensing device and the wearable sensing device is incorporated into the wearable. Again, the term “wearable” is intended to mean clothing, clothing portions, such as arm sleeves and leg sleeves, joint sleeves, joint wraps, torso coverings, other wraps, any type of removable patches, including both reusable and disposable patches that are attachable using medical grade adhesives, arm slings, knee braces, protective walking boots and other recuperative medical supports and braces.

Because each sensor alluded to above functions differently, placement of the sensor within the wearable sensing device or the wearable must be such that the sensor can actually “sense” the parameter or parameters that it is intended to sense. For example, the stretch of a sleeve which has conductive fibers woven into it requires only that the circumference of the sleeve be monitored in a relative fashion—that the sleeve be stretched at one or more points along the sleeve. A sensor used to measure the reflectivity of a patient's skin cannot be woven into such a sleeve in such fashion. Instead, the sensor must be at or near the surface of the wearable such that the sensor is able to sense the patient's skin color, as compared to a baseline. Such a sensor could also be incorporated into a sleeve or a patch that is worn over only a portion of the patient's skin. The point here is that the sensor(s) must be incorporated into the wearable(s) such that sensor functionality is not compromised such that the sensor is incapable of functioning as intended—and this is true for each type of wearable as it relates to each type of sensor.

It is also clear that any sensor that is incorporated into the wearable sensing device or the wearable must be capable of electronically communicating the parameters that the sensor is detecting and monitoring—as it is detecting them in real time. This is accomplished by some sort of “connectivity” between the sensor, which is preferably a MEMS-type unit that imparts an electrical signal the magnitude of which may be directly proportional to the “change” in the parameter being monitored, as analog or digital signals, and a processor. This “connectivity” allows the sensor to report physical and physiological parameter measurements to the processor, which processor is also preferably secured within the wearable sensing device or separately within the wearable. Further processing of the physical and physiological parameter measurements detected by the sensor may, however, be further processed by another centralized processor, as will be apparent later in this detailed description.

Each wearable sensing device preferably uses low energy digital technology and BlueTooth®, iBeacon™ or other short-wavelength ultra-high frequency (or “UHF”) radio wave technology in the industrial, scientific and medical (or “ISM”) band ranging from 2.4 to 2.485 GHz (BLUETOOTH is a registered certification mark of Bluetooth Sig, Inc. and IBEACON is a trademark of Apple Inc.); radio frequency (“RF” and “RFID”) technology; and/or other electronic data messaging modalities to send the monitored data to a receiver, a personal computing device, a smartphone, a terminal (as defined below) or to an electronic medical record (“EMR”) for the user patient. This functionality is consistent with the concepts of “telemedicine” and “telehealth.” The term “telemedicine” can be defined as the use of medical information exchanged from one site to another via electronic communications to improve a patient's clinical health status. Telemedicine includes a growing variety of applications and services using two-way video, email, smart phones, wireless tools and other forms of telecommunications technology. The term “telehealth” is sometimes used to refer to a broader definition of remote healthcare that does not always involve clinical services. Telemedicine is closely allied with the term health information technology (“HIT”). However, HIT more commonly refers to electronic medical records (“EMR”) and related information systems while telemedicine refers to the actual delivery of remote clinical services using technology, which can also be referred to as digital health. Both methods, however, must be compliant with the Health Insurance Portability and Accountability Act of 1996 (“HIPAA”).

Concerning the mediation aspect of the present invention, it complements the monitoring of certain physical and physiological parameters as described above. For example, one such monitoring and mediating concept in accordance with the present invention would comprise the following steps. First, a sleeve or brace is placed around the patient's limb (the “surgical limb,” which could include a single limb or multiple limbs could be involved, such as an upper extremity and a lower extremity, or some combination thereof) immediately following surgery. The sleeve or brace is a “wearable” comprised of at least one wearable sensing device and sensing element, as previously described. Next, specific baseline measurements of the surgical limb are taken immediately following surgery. Alternatively, the same measurements from the non-surgical limb could also be used as a baseline. In either approach, the baseline measurements are sent, via electronic signal, to a processor or stored in the memory of an individual wearable sensing device. Throughout the patient's post-surgical course, limb circumference measurements are taken and processed. If the limb circumference of the surgical limb reaches a certain point of swelling, which point exceeds an acceptable level of post-operative swelling, mediation will be initiated. Such mediation would be to reduce swelling or reduce the risk of blood clots by using intermittent pneumatic compression or sequential compression of the type described and claimed in this inventor's co-pending Patent Cooperation Treaty Application PCT/US2015/36920 titled Intermittent and Sequential Compression Device and Method, the content of which is incorporated herein by reference. This would also trigger messages to medical providers alerting them that the patient may be at risk of a blood clot in the surgically invaded limb. All recorded and stored electronic data relative to this monitoring and mediation can be used to populate the patient's EMR for later reference during and following treatment, again, in full HIPAA compliance.

Another example comprises the steps of placing a brace, sleeve or adhesive unit with a sensor on the patient's limb, as in the above example, but where EMG technology is used. As discussed above, EMG is a technique for measuring the electrical potential generated by muscle cells and detecting the activation level or electrical potential of such cells. Detection of the activation level or electrical potential comprises the use of a muscle measurement device such as the electromyography technology that likewise monitors limb circumference (other parameters can be included as well). The EMG or limb circumference features are used to monitor and detect a decrease in limb circumference monitored by the sensor which could be an indication that the patient's muscle isn't “firing” or is getting weaker. This will activate a muscle stimulation unit (could be built into the brace or by itself) that will help the muscle to fire or to decrease post-surgical muscle atrophy. All of the data relative to the patient's post-operative monitoring is recorded and stored in an on-board or remote memory, which could then be with a physical therapist who would devise and implement a plan for improving the patient's biomechanics. Here again, all recorded and stored electronic data relative to this monitoring and mediation can be used to populate the patient's EMR for later reference during and following treatment, all in full compliance with HIPAA.

Yet another example would be to apply a joint sleeve and brace to a post-surgical or injured joint. Certain embedded range of motion (“ROM”) sensors can monitor the progress (or lack of progress) being made in physical therapy of joint ROM. Limb circumference measurements and EMG can also track muscle atrophy and activity. Based on these parameters, the physical therapist can adjust exercises. Alternatively, a mobile application (or “app”) can be used to customize a rehab protocol based on joint function and muscle activity received. This data could also be used to determine when a patient could remove a brace or get off crutches. Here again, all recorded and stored electronic data relative to this monitoring and mediation can be used to populate the patient's EMR for later reference and to follow progress of recovery. The app will be discussed later in this detailed description.

It is to be understood that the examples of monitoring a patient's physical and physiological parameters and then mediating any medical abnormalities following surgical intervention and/or assessing and treating certain patient biomechanics is not limiting of the present invention. Multiple combinations can be made for such purposes, all of which are within the scope of the present invention.

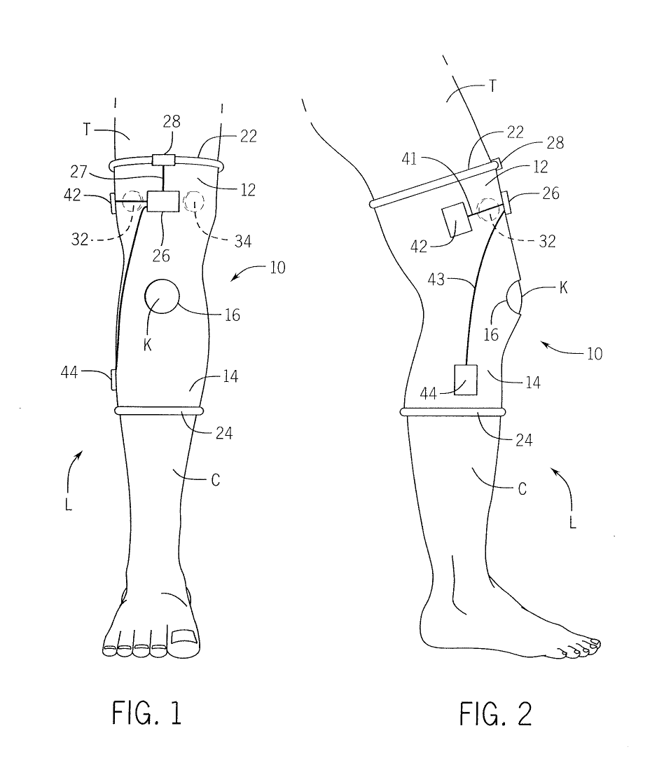

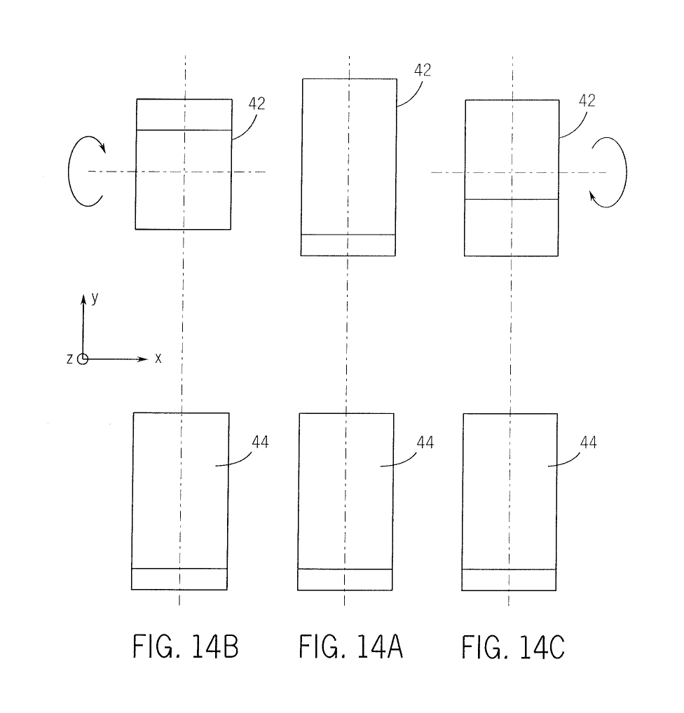

Referring now to the drawings, wherein like numbers and letters represent like structure throughout, FIG. 1 shows a first exemplary embodiment of a leg wearable, generally identified 10, that is constructed in accordance with the present invention. The wearable 10 is fabricated from synthetic fibers, or a combination of synthetic fibers and natural fibers. The desired characteristic of the wearable 10 is that it be flexible and stretchable so as to closely fit the contours of the patient's limb. In other instances, the wearable 10 can comprise a brace or similar structure such as one disclosed and claimed in this inventor's previously mentioned co-pending Patent Cooperation Treaty Application. The term “synthetic fiber” can be construed to include man-made fibers such as polyesters, acrylics, nylon, rayon, acetates, among others. The precise type of fabric is not a limitation of the present invention. In the case where the wearable 10 is a mechanical brace of the type shown in FIG. 14 of this inventor's co-pending Patent Cooperation Treaty Application PCT/US2015/36920 titled Intermittent and Sequential Compression Device and Method, incorporated here by reference, the brace may also be fabricated of plastic materials and metal.

Referring to both FIGS. 1 and 2, the patient's leg L is shown to comprise a thigh T, knee K and calf C. The leg wearable 10 is a substantially tube-shaped structure that encircles the patient's leg L and comprises an upper portion 12 and a lower portion 14, the upper portion 12 having a slightly larger diameter that that of the lower portion 14. The upper portion 12 is disposed above the patient's knee K and about the patient's thigh T. The lower portion 14 is disposed below the patient's knee K and about the patient's calf C. This allows for form fitting of the wearable 10 about the thigh T, knee K and calf C when worn as intended. An aperture 16 is provided to the anterior of the wearable 10 to prevent fixation of the knee cap during normal flexing of the knee K during use of the wearable 10, the existence and size of which is an optional design expediency.

The upper portion 12 comprises a circumferential band 22 of a fixed length, the ends of the band 22 being connected to a stretch sensor 28. The stretch sensor 28 essentially measures the distance between the band ends (not shown) when the band 22 is pulled by tissue expansion. Alternatively, the band 22 could also comprise a plurality of longitudinally-extendable wires woven into the fabric of the wearable 10, or other design expediencies that would allow for the detection of changes in limb circumference at that point of the patient's thigh T. The lower portion 14 similarly comprises a similar structure 24. Other placements for the circumferential sensors 22, 24 are within the scope of the present invention. As shown in FIG. 1, the stretch sensor 28 is electrically connected to a stand-alone processing unit 26 via a combination communication and power input/output wire 27. It should be noted here that, in this particular embodiment, the battery 26B of the processing unit 26 provides a DC power supply for each of the other electrically-powered components of the processing unit 26. See FIG. 16. The processing unit 26 is disposed to the anterior outer surface of the wearable 10. This unit 26 also includes a microprocessor 26A and a transmitter 26C, the latter of which is provided to wirelessly transmit signals 29 via an antenna 28 based on the sensed parameters being processed by the various sensors used in the wearable 10. Again, see FIG. 16.

In this first exemplary embodiment, a pair of EMG monitors 32, 34 are provided, but are disposed at the interior surface of the wearable 10, the EMG monitors 32, 34 requiring juxtaposed positioning relative to the patient's skin to measure, for example, quadrilateral muscle contraction in the distal quadrilateral muscle of the patient's thigh T. The EMG monitors 32, 34 are also electrically connected to the processing unit 26. The wearable 10 also comprises an upper wearable sensing device 42 and a lower wearable sensing device 44, each of which is electrically connected via combination power and communication wires 25, 23, respectively. Each wearable sensing device 42, 44 includes ten axes of motion—three via a gyrometer sensing element, three via a magnetometer sensing element, three via an accelerometer sensing element and one via a barometric sensing element—the functionality of which will be discussed later in this detailed description as will the significance of their positioning above and below the knee K. However, it is to be understood that other sensors, such as sensors having other multiple axes of motion (three, six or nine) could be included with this wearable 10 and that such is not a limitation of the present invention. Further, it is to be understood that the placement of the wearable sensing devices 42, 44 is not limited to the precise position shown in FIGS. 1 and 2. That is, the wearable sensing devices 42, 44 could be disposed farther apart from one another, could be placed on the wearable to the ventral portion or front of the leg L, to the dorsal portion or back of the leg L or even at a point to the inside of the leg L, although the latter is likely to prove impractical as it holds the most potential for the sensing device 42, 44 to be brushed against by the other leg (not shown) and then misaligned or unaligned from its original position.

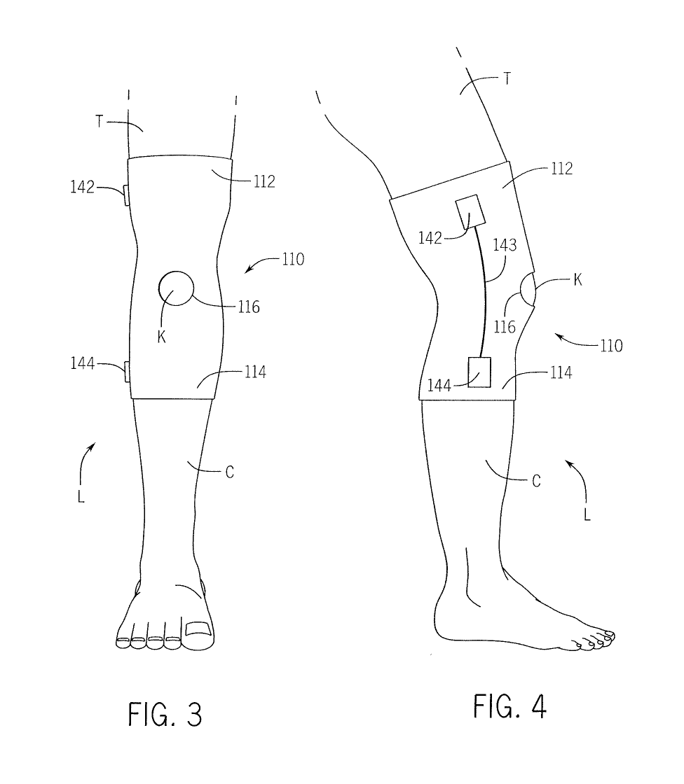

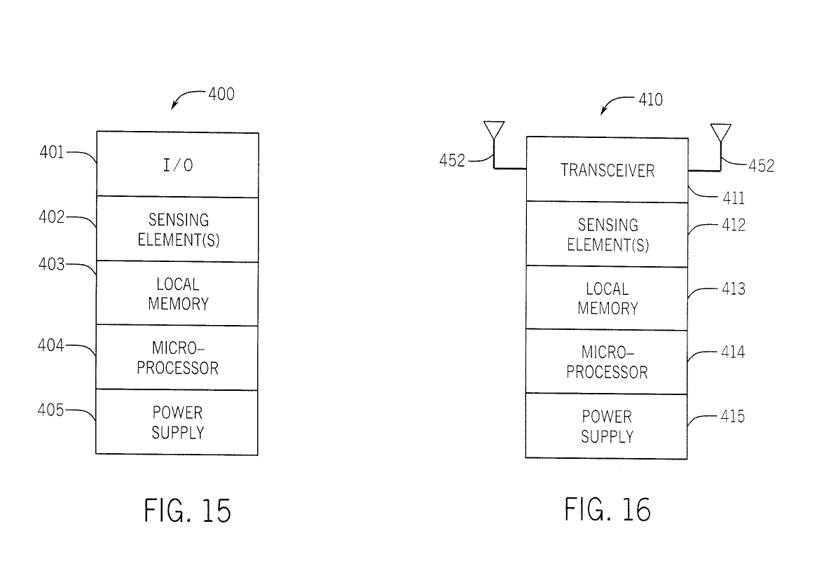

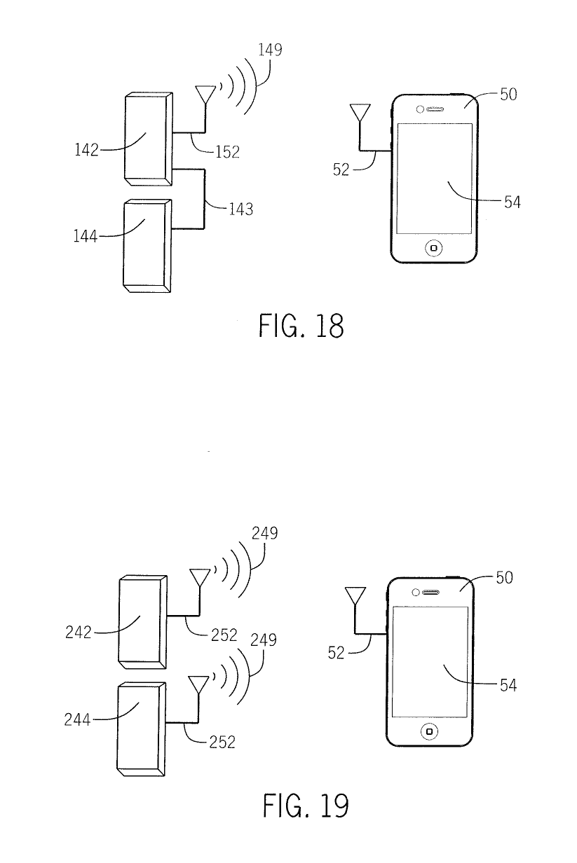

Referring now to FIGS. 3 and 4, they show a similar wearable 110 that is also constructed in accordance with the present invention. In this embodiment, the wearable, generally identified 110, comprises an upper portion 112 and a lower portion 114. An upper wearable sensing device 142 is provided as is a lower wearable sensing device 144, one above the knee and one below it, respectively. In this embodiment, the MEMS configuration 400 for the lower wearable sensing device 144 comprises an on-board power supply 405, a microprocessor 404, a local memory 403 (which can be transitory or non-transitory memory), ten axes sensing elements 402 (plus others, as required) and an input/output component 401, all contained within a single housing. See FIG. 15. The MEMS configuration 410 for the upper wearable sensing device 142 comprises an on-board power supply 415, a microprocessor 414, a local memory 413 (which can also be transitory or non-transitory memory), ten axes sensing elements 412 (plus others, as required) and a transceiver 411 having at least one wireless antenna 452, also contained within a single housing. The upper and lower wearable sensing devices 142, 144 and connected via a combination power and communication wire 143. In this way, only one of the wearable sensing devices, in this case the upper wearable sensing device 142 requires wireless connectivity to a personal computing device 50. See FIG. 18.

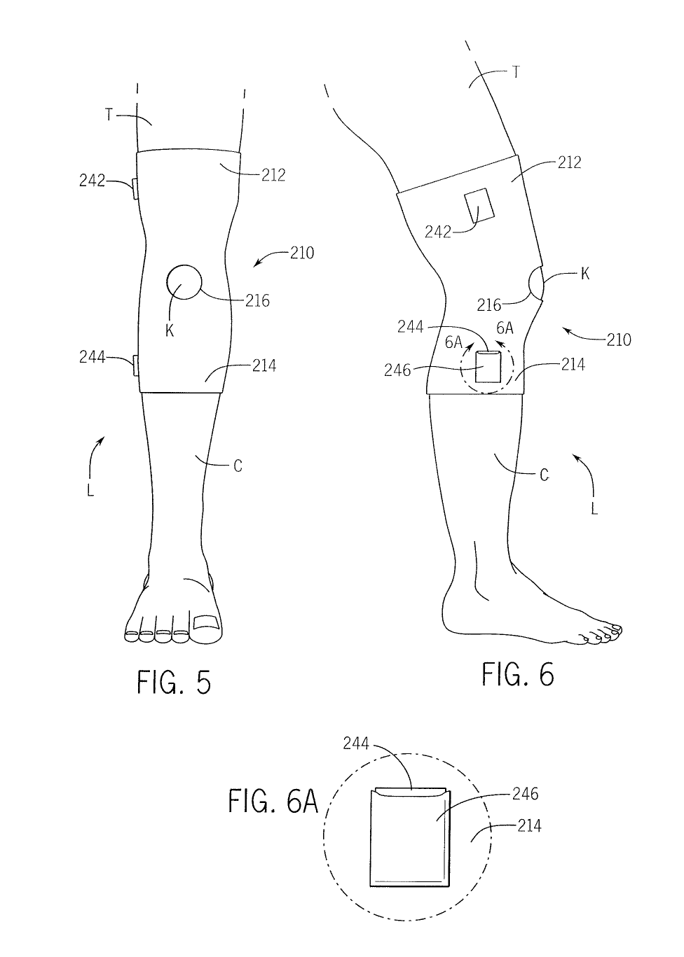

Referring now to FIGS. 5 and 6, they show another wearable 210 that is also constructed in accordance with the present invention. In this embodiment, the wearable, general identified 210, comprises an upper portion 212 and a lower portion 214. An upper wearable sensing device 242 is provided as is a lower wearable sensing device 244, one above the knee and one below it, respectively. With reference to FIG. 6A, it is to be appreciated that any sensing device, including the lower wearable sensing device 244 shown, can, and preferably is, held in position via a pocket 246 that is formed into the wearable 210. The pocket 246 is sized such that the sensing device 244 is firmly held in position during use. Absent a firm fit, the wearable sensing device 244 could move around within the pocket 246, resulting in the acquisition of inaccurate positional information by the sensing device 244. In this particular embodiment, the MEMS configuration 500 for the upper and lower wearable sensing devices 144, 142 is the same—an on-board power supply 415, a microprocessor 414, a local memory 413, ten axes sensing elements 412 and a transceiver 411 having at least one wireless antenna 252, also contained within a single housing. See FIG. 16. In this way, both wearable sensing devices 242, 244 are wirelessly connected to a personal computing device 50. See FIG. 19.

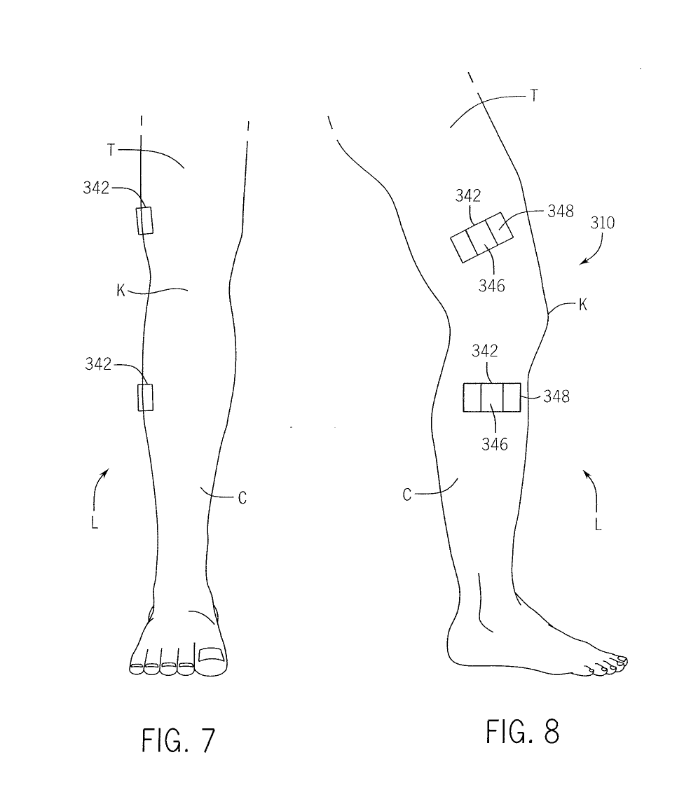

Referring now to FIGS. 7 and 8, they show yet another type of wearable sensing device, generally identified 310, in the form of a patch or bandage (a “patch or bandage wearable” and a “patch or bandage wearable sensing device”) that adheres to the patient's limb at pre-determined optimal positions. This embodiment is significant in that it allows for use of ten axis sensing without the need for wearing a full sleeve or brace. However, it would also be within the scope of the present invention to have the patch or bandage wearable 342 attached to an existing brace of current manufacture without any retrofitting or interference with the functionality of the brace, which functionality would remain intact. In this example, one patch or bandage wearable sensing device 342 is disposed above the patient's knee K and one patch or bandage wearable sensing device 342 is disposed below the knee K. Adhesion to the skin of the user is accomplished by means of an adhesive strip 348 that is disposed atop of and to either side of a wearable sensing device 342. An alternative attachment means would be to place an adhesive (not shown) to the skin-side of the patch or bandage wearable sensing device 342. This adhesive would be covered by a removable strip which, when removed, exposes the adhesive and allows the device 342 to be attached. Either construct allows for variable placement of the patch or bandage wearable sensing device 342 in virtually any location that is desired or required. The encapsulation of the MEMS components within the sensor housing is the same as that of the previous embodiment described relative to FIGS. 5 and 6. In this way, both patch or bandage wearable sensing devices 342 are wirelessly connected to a personal computing device 50. Again, see FIG. 19.

Significantly, the positioning of the ten axis wearable sensing devices 42, 44, 142, 144, 242, 244, 342 and the sensing elements 402, 412 contained within each allows for the measurement of the patient's ROM at the knee K. They also allow for the detection of joint alignment at the knee (or knees), such as varus (bowed legs) and valgus (knock-knee) conditions. The wearable sensing devices 42, 44, 142, 144, 242, 244, 342 and the sensing elements 402, 412 contained within each can also measure how many times the knee K has flexed, how many times the patient has kneeled, and so on. Further, the wearable sensing devices 42, 44, 142, 144, 242, 244, 342 and the sensing elements 402, 412 contained within each, or within any one of them, can be used to monitor normal activity tracking metrics, such as distance traveled, number of steps taken, number of stairs climbed, calories expended, and the like.

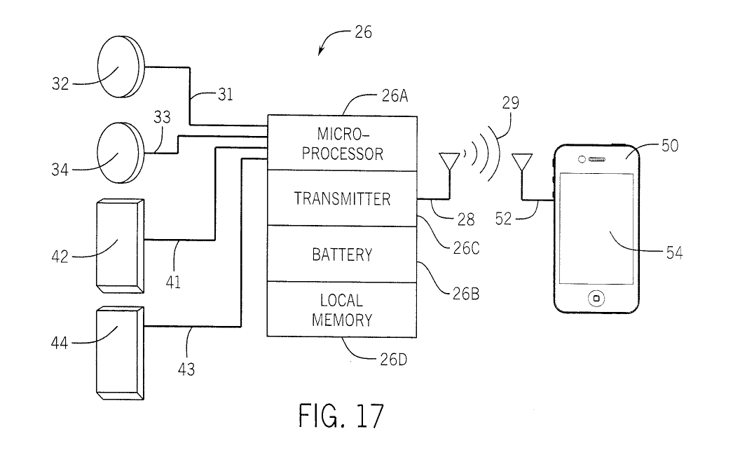

Referring now to FIG. 9, it shows a pair of ten axis wearable sensing devices 42, 44 of the type described above. Each wearable sensing device 42, 44 comprises a housing 45, 47 and a tapered leading nose or face 46, 48, which serves as a visual aid to facilitate proper orientation of the devices 42, 44. The noses 46, 48 also serve as a physical aid upon insertion of the ten axis wearable sensing devices 42, 44 into a sleeve, as previously described, by making it easier for the user to insert the noses 46, 48 into the sleeve, which is required to be somewhat of a tight fit to insure the desired or required positioning of the devices 42, 44 is maintained. Following initial placement of the ten axis wearable sensing devices 42, 44, the relative position of the ten axis wearable sensing devices 42, 44 is calibrated by one of the sensing devices—either of which can be used for that purpose. Once calibrated, movement of the first wearable sensing device 42 as compared to the dynamic comparable positioning of the second wearable sensing device 44 is sensed by the second wearable sensing device 44 in an x, y and z Cartesian coordinate grid (collectively, the “ten axis sensors”). As shown, the position of the first wearable sensing device 42 is above the second wearable sensing device 44, much the same as the wearable sensing device 42. 44 would be positioned above and below a patient's knee K as shown in FIG. 1. Thereafter, spatial location, dynamic movement, including rotation rates, angular velocities, acceleration and deceleration can be detected via the sensing elements 402, 412 and differentials measured via the on-board microprocessors 404, 414, respectfully, and inputted into on-board local memories 403, 413. That is, rotation of the sensing element 402, 412 (depending on whether the wearable sensing device is a hard wired version or a wireless version) is detected with respect to the x, y and z Cartesian coordinate grid axis the rotation rate and angular velocity. The angular velocity includes three components corresponding to the rotation rate or angular velocities of the sensor about each of the first axis (x), the second axis (y) and the third axis (z). Similarly, acceleration and deceleration of the sensing element is detected with respect with to the grid and includes three components corresponding to the acceleration or deceleration about the first axis (x), the second axis (y) and the third axis (z). The microprocessors 404, 414 can conduct sampling of such rates and other motion measurements to provide data from the sensing element 402, 412 or to a centralized microprocessor 26A, as shown in FIG. 17.

As mentioned at the outset, the algorithmic steps that are made in accordance with the present invention utilize applied “quaternion” matrix math, which is used to determine a rotation angle and the vectored direction of a rotation. A quaternion is technically four numbers, three of which have an imaginary component. The quaternion itself is defined as q=w+xi+yj+zk where w, x, y, and z are all real numbers and i, j and k are imaginary numbers. The imaginary numbers are not particularly important from a programming perspective. The number w is the amount of rotation about the axis defined by <x, y, z>. The magnitude of a quaternion is given by the formula magnitude=square root of (w²+x²+y²+z²). The primary practical application of quaternions is to represent three-dimensional rotations.

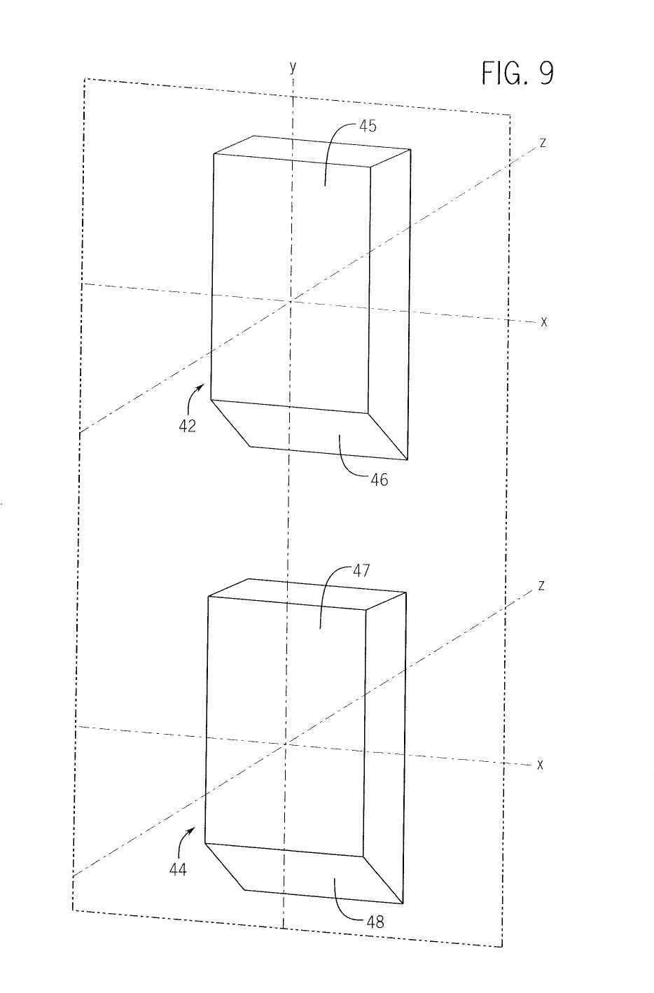

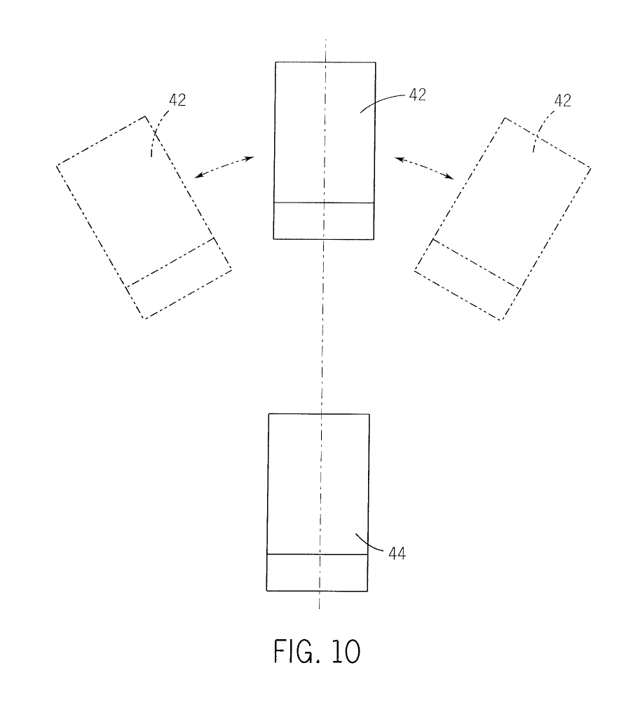

Referring now to FIG. 10, it shows how “relative” movement between the wearable sensing devices 42, 44 shown in FIG. 9 (the upper wearable sensing device 42 being disposed along the user's femur and experiencing less movement than the lower wearable sensing device 44, which is disposed along the user's tibia) effectively results in a rotation of one wearable sensing device 42 relative to the other wearable sensing device 44. This translates directly into the “dueling” motion detection between the sensing elements 402, 412 that are contained within the wearable sensing devices 42, 44, the position of such sensing elements 402, 412 being fixed within the MEMs circuitry of the wearable sensing devices 42, 44 irrespective of such positioning, which is always going to be “relative.”

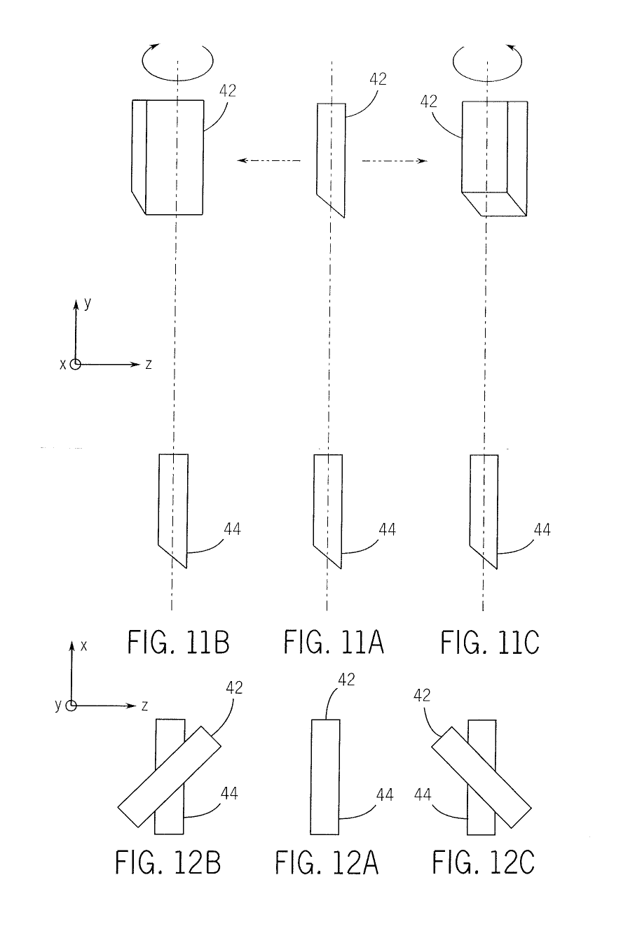

For simplicity, it is to be assumed that the wearable sensing devices 42, 44 are attached or positioned relative to a joint (as when, for example, the upper wearable sensing device 42 is secured above a knee joint and the lower wearable sensing device 44 is secured below the knee joint) which results in a relative rotation of the upper wearable sensing device 42. Also for simplicity, it is to be assumed that the wearable sensing devices 42, 44 are disposed on a surface (i.e. the outer surface of a user's right leg) in a substantially coplanar fashion. See, for example, FIG. 11A. The rotational movement shown in FIG. 10 would include the parameters of (i) linear and rotational directions, (ii) dynamic linear and rotational speeds and (iii) dynamic linear and rotational accelerations or decelerations. In accordance with the previously described functionality of the wearable sensing device 42, 44, these parameters are detected by the sensing elements 402, 412 and parameter values are then manipulated by implementing the algorithmic steps relating to those parameter values via the microprocessors 404, 414. This allows for the extraction of useful bioinformatics from large amounts of raw data provided by the sensing elements 402, 412 within each wearable sensing device 42, 44, such bioinformatics then being provided to the user or healthcare provider via a feedback component as desired or required. As will be discussed to a greater extent later in this detailed description, the bioinformatics can be processed instead via the user's personal computing device 50 on which resides the computer processing program.

Referring now to FIGS. 11A, 11B and 11C, they show the same wearable sensing device 42, 44 as are shown in FIGS. 9 and 10, with one wearable sensing device 42 being disposed above the other wearable sensing device 44. In this example, the upper wearable sensing device 42 is rotated about the y axis in relation to the lower wearable sensing device 44, as would be the case where the wearable sensing devices are placed in vertical positions to either side of a joint and the joint is showing some degree of rotation, albeit the degree of rotation shown is greatly exaggerated. FIGS. 12A, 12B and 12C are top plan views showing the shifting of the upper wearable sensing device 42 relative to the stationary lower wearable sensing device 44. In this example, the parameters are likewise detected and parameter values are similarly manipulated by implementing the algorithmic steps relating to those parameter values. This allows for the extraction of additional useful bioinformatics relative to the joint in issue—specifically, whether the joint is showing “twist.”

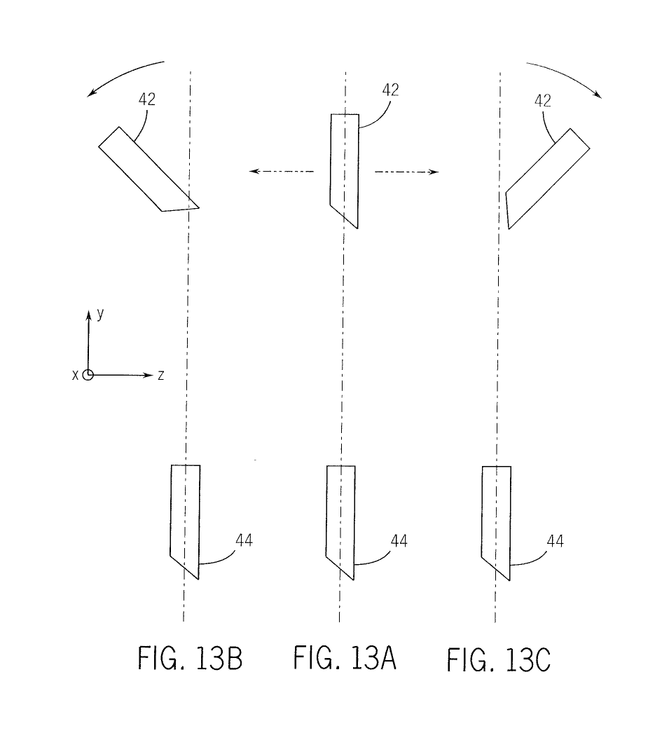

Referring now to FIGS. 13A, 13B and 13C, they show the same wearable sensing devices 42, 44 as are shown in FIGS. 9 and 10, with one wearable sensing device 42 being disposed above the other wearable sensing device 44. In this example, the upper wearable sensing device 42 is rotated about the x axis in relation to the lower wearable sensing device 44, as would be the case where the wearable sensing devices are placed in vertical positions to either side of a joint and the joint showing exaggerated valgus and varus joint alignments. Again, this degree of rotation is greatly exaggerated. FIGS. 14A, 14B and 14C are views that correlate to those shown in FIGS. 13A, 13B and 13C when viewed from the z-axis. Here again, the positional parameters are likewise detected and parameter values are similarly manipulated by implementing the algorithmic steps relating to those parameter values. This allows for the extraction of further useful bioinformatics relative to the joint in issue—specifically, whether the joint is showing “twist.”

In summary, the positional parameters shown in the above-referenced drawings are, for purposes of understanding the “dueling sensor” concept in the device and method of the present invention, exemplary only.

As they relate to a knee joint, for example, the measured parameters could include ROM (flexion/extension), where a loss of motion may predict arthritis or cartilage damage; joint rotation, where excessive rotation may increase the risk of cartilage tears; joint alignment (varus/valgus), where increasing varus or valgus is another indicator of arthritis in the joint; ligament laxity, where excessive translation of the tibia indicates the tear of an anterior cruciate ligament (“ACL”), an excessive tibia and femur gap on the inner (medial) or outer (lateral) joint line can indicate tearing of the medial collateral ligament (“MCL”) or the lateral collateral ligament (“LCL”), or how well a ligament surgery was done; time spent with knee flexed more than 90°, with more time indicating increased risk of knee cap pain; number of times the knee is flexed more than 90°, which can indicate the risk of knee cap pain; and number of times the knee is cycled per day (i.e., going from flexion to extension and back), which can be used to predict survival time in years of knee replacement.

As they relate to an elbow joint, for example, the measured parameters could include ROM (flexion/extension), where decreased motion indicates arthritis or muscle damage; amount of gaping on inner elbow, which can indicate that the ulnar collateral ligament (“UCL” or “Tommy John” ligament) is torn; stress on ulnar collateral ligament, which can be a predictor of risk of injury to UCL; and forearm rotation (pronation/supination), a decrease of which can indicate arthritis or muscle/ligament damage.

It is to be understood that other joints, in addition to the knee and elbow joints discussed above, are well within the scope of the device and method of the present invention. For example, joints involving the cervical spine (or neck), lumbar spine (or lower back), wrist, hip, ankle and the like are joints with which the device and method of the present invention could be used. Further, and as wearable sensing devices and sensors of the type disclosed and claimed herein are made smaller and smaller, all potential joints could be monitored for similar parameters.

Referring now to FIGS. 17 through 19, they illustrate electronic configurations for three exemplary embodiments of the wearable system in accordance with the present invention. FIG. 17 shows the wearable sensing devices 42, 44 being hard wired to the onboard battery and a processing unit 26 in accordance with the device configuration illustrated in FIGS. 1 and 2. The processing unit 26 is, in turn, wirelessly connected to a personal computing device 50 having a user interface 54 in the form of a monitor or touch screen, as is well known in the art. FIG. 18 shows the wearable sensing devices 142, 144 wired together via a communication line 143 and the upper wearable sensing device 142 comprising an antenna 152—only one wearable sensing device 142 being wirelessly connected to the personal computing device 50—and the antenna 152 emitting an electromagnetic wave signal 149. FIG. 19 shows each of the wearable sensing devices 242, 244 having an antenna 252 that emits an electromagnetic wave 249, making the wearable sensing devices 242, 244 wirelessly connected to the personal computing device 50.

Referring back to FIG. 17 it shows the EMG monitors 32, 34 being connected to the programmable logic controller (“PLC”) 26A of a processing unit 26 via wire leads 31, 33, respectively. The ten axis wearable sensing devices 42, 44 are similarly connected via wire leads 41, 43, respectively. It is to be understood that the wire leads 31, 33, 41, 43 could be imbedded into the wearable 10 or surface mounted in some fashion known in the art. It is also to be understood that the wire leads 31, 33, 41, 43 can be strictly communication signal wires or, alternatively, combined communication signal wires and direct current power transmission wires for the various sensors and/or wearable sensing devices. The processing component 26 comprises the microprocessor 26A, an onboard battery 26B, a wireless transmitter 26C and a memory component 26D, which can be transitory or non-transitory memory. The microprocessor 26A is provided to implement the algorithmic steps and instructions relative to any monitored parameter. The onboard battery 26B is preferably a lithium ion battery of the type that can recharge via induction charging. However, the battery 26B could also be the type that is replaceable or rechargeable via “plug-in” charging technology. The wireless transmitter 26C comprises a transmission antenna 28 that is capable of emitting electromagnetic waves 29 that carry wireless communication signals. In this particular embodiment, it will be seen that a portable computing device 50 comprises a receiver antenna 52 for receiving the wireless communication signals, the signals then being inputted and then processed by the processor (not shown) that is housed within the portable computing device 50 and then displayed via the monitor 54. This can be done in real time or via a memory component (not shown) contained within the portable computing device 50. Although the circumferential wearable sensing device 22, 24 are not shown in FIG. 17, it is to be appreciated that the same type of electrical connection to the processing component 26 is within the scope of the present invention, as are other types of sensors and monitors of the type discussed elsewhere in this detailed disclosure.

As alluded to previously, the information and data detected and measured by any of the sensors mentioned above can be transmitted to a portable computing device using various short and long range wireless communication means, as described in greater detail below. Specifically, this inventor contemplates the use of the sensors in combination with a wireless device, such as a smartphone, cell phone or the like via a mobile application (or “app”). The app can function in a myriad of ways, for a wide range of applications and with a wide range of personal computing devices or other wireless device.

By way of specific example relative to the knee joint previously discussed, the app can process information and data parameters relating to the knee joint. The app could monitor normal activity tracking functions such as distance traveled, calories burned, flights of stairs climbed, time spent standing, time spent sitting, and so on. Further, the app could predict the risk of certain knee injuries. The app could also monitor certain “predictables,” such as predicting and diagnosing causes of knee pain, and give exercises and tips for decreasing injury risks, or other “trainables.” Strategies and exercises to decrease knee pain could be provided, including coaching tips to improve performance. The app could also allow a healthcare provider to monitor a patient after an injury or surgery and alert them if patient restrictions have been exceeded, with alerts also being provided to the patient. Progress following a diagnosed injury or following surgery could product a 3-dimensional live time rendering of a knee joint with ROM data and showing a heat map to determine where more or less stress is being placed on the joint. The app could also allow for telemedicine physical exams by a physician or allow for home rehabilitation under the supervision of a therapist. The app could also forward a joint exam to a patient's EMR so that the physician will not have to repeat an exam, which can decreased office time and allow for more efficient uses of time with patients.

By way of another specific example relative to the elbow joint previously discussed, the app can process information and data parameters relating to that joint as well. For example, the app could monitor elbow ROM, forearm rotation, stress on UCL, predict risk of UCL tear, alert patients of exceeded limits on motion post-surgery, predicts and diagnose causes of elbow pain and provide rehab exercises and tips to decrease elbow pain. By way of a specific example relating to the elbow in an athletic setting, the app could include a pitch/throwing counter for baseball players, predict how a fastball was thrown and give coaching tips to decrease UCL stress.

Among other things, the app could also inform a user as to a reason that the user is having pain based on current data and information detected via the sensor(s). Sensor data can also provide feedback to physical therapists and physicians who are tracking patient progress and performance following an injury or surgery. Such data and information could also be used with patients who undergo joint replacements. In the MEMS of the present invention, feedback via a vibratory off-set cam motor could let a patient know when limits have been reached or when positioning is wrong. This would allow the user to take corrective action on his or her own as needed.

Continuing, and for purposes of further illustrating enablement of the present invention, it is also to be generally understood that the wearables 10, 110, 210, 310 and the electronic functionalities discussed with respect to them and the wearable sensing device sensors in particular, are configured to “interface” with a wide variety of data “terminals,” including, but not limited to, iPhone® devices (iPhone is a registered mark of Apple, Inc.). Such terminals include mobile as well as stationary terminals, including mobile phones, smart phones, computers, digital broadcast terminals, personal digital assistants and portable multimedia players. Further description will be with regard to a mobile terminal, but it should be noted that such teachings apply equally to other types of electronic terminals. The mobile terminal in accordance with the preferred embodiment of the present invention can include a wireless communication unit, an input unit, an output unit, a memory (which, again, can be a transitory or a non-transitory memory), an interface unit, a controller, a power supply unit and the like. It is also to be understood that implementing all of the components is not a requirement as greater or fewer components may be implemented. Further, all recorded and stored electronic data relative to any monitoring and mediation used in accordance with the present invention can be used to populate the patient's EMR for later reference during and following medical treatment. This would apply to a more centralized server or terminal that is used for such purposes.

The wireless communication unit that is used with the present invention includes one or more components which permit wireless communication between the monitoring device and a wireless communication system or network, such as an EMR database. For instance, the wireless communication unit can include a broadcast receiving module, a mobile communication module, a wireless internet module and the like. The broadcast receiving module receives a broadcast signal and/or broadcast associated information from an external broadcast managing server via a broadcast channel. The broadcast channel may include a satellite channel and a terrestrial channel. The broadcast managing server generally refers to a server which generates and transmits a broadcast signal and/or broadcast associated information or a server which is provided with a previously generated broadcast signal and/or broadcast associated information and then transmits the provided signal or information to a terminal. The broadcast associated information includes information associated with a broadcast channel, a broadcast program, a broadcast service provider, etc. The broadcast associated information can be provided via a mobile communication network. In this case, the broadcast associated information can be received by the mobile communication module. The broadcast associated information can be implemented in various forms. For instance, broadcast associated information may include an electronic program guide of digital multimedia broadcasting and electronic service guide of digital video broadcast-handheld. The broadcast receiving module may be configured to receive broadcast signals transmitted from various types of broadcast systems. The broadcast signal and/or broadcast associated information received by the broadcast receiving module may be stored in a suitable device, such as a memory. The mobile communication module transmits/receives wireless signals to/from one or more network entities (e.g., base station, external terminal, server, etc.). Such wireless signals may represent audio, video, and data according to text/multimedia message transceivers, among others. The wireless internet module supports Internet access for the mobile terminal. This module may be internally or externally coupled to the mobile terminal. In this case, the wireless Internet technology can include Wireless LAN, Wi-Fi, Wibro, Wimax, HSDPA, etc.

A position-location module could also be utilized in the present invention, the position-location module being functionally adapted to identify or otherwise obtain the location of the mobile terminal. This module may be implemented with a global positioning system (“GPS”) module, as previously discussed, and would be particularly useful with sensors that monitor positional relationships within certain wearables.

The input unit generates input data responsive to monitored physical and physiological parameters of the patient. In the present invention, the user input unit is physical and physiological parameters that have been monitored, measured and then downloaded in real time or following recordation and synchronization of the physical or physiological parameter data contained within the memory of an on-board device. The output unit generates outputs relevant to one or more of the monitored parameters. Further, the output unit would include a display such as a visual display that would show the user or the healthcare provider the monitored results. For example, the display will generally provide a user interface (“UI”) or graphical user interface (“GUI”) which includes information associated with the monitored parameters. As another example, the display may additionally or alternatively display images which are associated with these modes, the UI or the GUI. The display module may be implemented using known display technologies including, for example, a liquid crystal display (“LCD”), a thin film transistor-liquid crystal display (“TFT-LCD”), an organic light-emitting diode display (“OLED”), a flexible display and a three-dimensional display. Some of the above displays can be implemented in a transparent or optical transmissive type, which can be named a transparent display. Where the display and a sensor for detecting a touch action (hereinafter called “touch sensor”) configures a mutual layer structure (hereinafter called “touchscreen”), it is able to use the display as an input device as well as an output device. In this case, the touch sensor can be configured as a touch film, a touch sheet, a touchpad or the like. The touch sensor can be configured to convert a pressure applied to a specific portion of the display or a variation of a capacitance generated from a specific portion of the display to an electric input signal. Moreover, it is able to configure the touch sensor to detect a pressure of a touch as well as a touched position or size. If a touch input is made to the touch sensor, a signal corresponding to the touch is transferred to a touch controller. The touch controller processes the signal and then transfers the processed signal to the controller. Therefore, the controller is able to know whether a prescribed portion of the display is touched. A proximity sensor can be provided to an internal area of the mobile terminal enclosed by the touchscreen or around the touchscreen. The proximity sensor is the sensor that detects a presence or non-presence of an object approaching a prescribed detecting surface or an object existing around the proximity sensor using an electromagnetic field strength or infrared ray without mechanical contact. Hence, the proximity sensor has durability longer than that of a contact type sensor and also has utility wider than that of the contact type sensor. The proximity sensor can include one of a transmissive photoelectric sensor, a direct reflective photoelectric sensor, a mirror reflective photoelectric sensor, a radio frequency oscillation proximity sensor, an electrostatic capacity proximity sensor, a magnetic proximity sensor, an infrared proximity sensor and the like. In case that the touchscreen includes the electrostatic capacity proximity sensor, it is configured to detect the proximity of a pointer using a variation of electric field according to the proximity of the pointer. In this case, the touchscreen can be classified as the proximity sensor. This type of proximity sensor is not to be confused with the physical and physiological parameter sensors that are the subject of the present invention.

The memory unit is generally used to store various types of data to support the processing, control, and storage requirements of the mobile terminal. The memory unit also includes the ability to collect data as to any of the monitored physical or physiological parameters. Further, a recent parameter history or a cumulative parameter frequency of each data (e.g., use frequency for each sensor input) can be stored in the on-board memory of the wearable or can be transmitted via wireless signal to a centralized server. In either case, the memory may be implemented using any type or combination of suitable volatile and non-volatile memory or storage devices including hard disk, random access memory (“RAM”), static random access memory (“SRAM”), electrically erasable programmable read-only memory (“EEPROM”), erasable programmable read-only memory (“EPROM”), programmable read-only memory (“PROM”), read-only memory (“ROM”), magnetic memory, flash memory, magnetic or optical disk, multimedia card micro type memory, card-type memory (e.g., SD memory, XD memory, etc.), or other similar memory or data storage device. Further, the memory can be transitory or non-transitory in nature and the mobile terminal is able to operate in association with web storage for performing a storage function of the memory on the internet, which would be a “cloud-based” web storage capability.

The controller, or microprocessor 404, 414, 26A, typically controls the overall operations of the wearable 10, 110, 210, 310 and its associated electronics. For example, the microprocessor 404, 414, 26A performs the control and processing associated with the monitored parameters. The microprocessor 404, 414, 26A may include a multimedia module that provides multimedia playback of the parameter data. The multimedia module may be configured as part of the microprocessor 404, 414, 26A, or implemented as a separate component. It should also be understood that various embodiments described herein may be implemented in any computer-readable medium using, for example, computer software, hardware, or some combination thereof. For a hardware implementation, the embodiments described herein may be implemented within one or more application specific integrated circuits (“ASIC”), digital signal processors (“DSP”), digital signal processing devices (“DSPD”), programmable logic devices (“PLD”), field programmable gate arrays (“FPGA”), processors, controllers, micro-controllers, microprocessors, other electronic units designed to perform the functions described herein, or a selective combination thereof. The point being that all of such embodiments may also be implemented by the microprocessor 404, 414, 26A.

For software implementation, the embodiments described herein may be implemented with separate software modules, such as procedures and functions, each of which perform one or more of the functions and operations described herein. Software implementation can be accomplished via software that is installed directly on the microprocessor 404, 414 of a wearable sensing device 400, 410, respectively. See FIGS. 15 and 16. See also the sensing device 26 as per FIG. 17. The software codes can be implemented with a software application written in any suitable programming language and may be stored in memory and executed by the controller or microprocessor 404, 414, 26A. That is, the software includes source code which is a list of instructions, written in a selected computer language, and then converted into computer machine language, which language the computer uses to build the software “machine” described by the instructions. The software machine is made up of the components referred to above. The source code is a detailed “blueprint” telling the computer how to assemble those components into the software machine. Further, the source code is organized into separate files, files are organized into separate modules, and modules are organized into separate functions or routines to accomplish, via pre-programmed algorithms, the necessary steps in accordance with the method and system of the present invention.

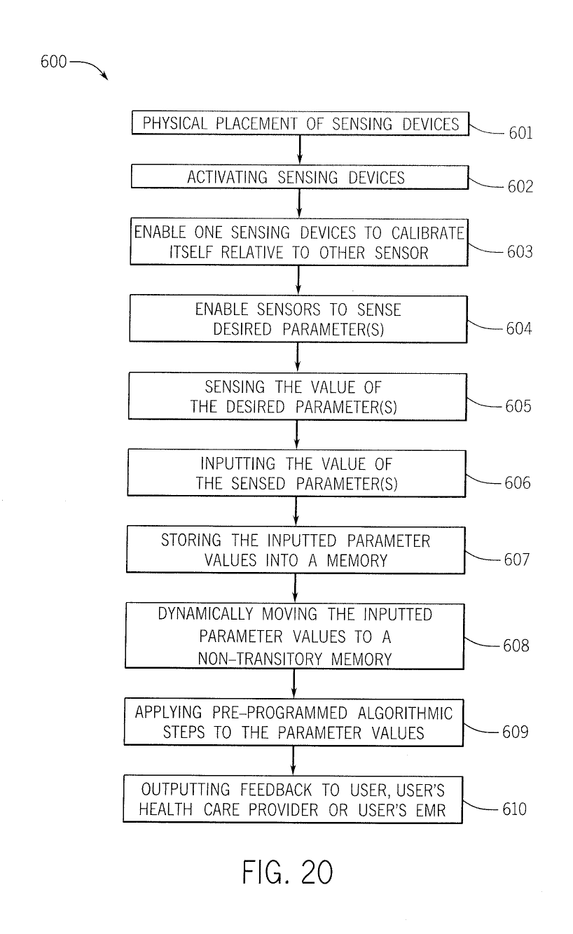

By way of example, FIG. 20 is a flow chart 600 illustrating the basic steps in processing physical or physiologic parameter data in accordance with the method of the present invention. As shown, the dueling wearable sensing devices are first placed into position 601, typically one to one side of a joint and another to the other side of a joint. The wearable sensing devices are then activated 602. One wearable sensing device is enabled to “calibrate” that sensing device relative to the other 603, which provides a starting or registration point for both sensing devices. This could be a location, a position, or the starting point for any physical parameter. Once calibrated, the sensors are enabled 604 for the purpose of sensing one or more desired physical parameters. Once the sensors sense 605 the parameter value, the parameter values are inputted 606 and then stored 607 in the transitory memory of the sensing device. The parameter values are then dynamically moved 608 to the non-transitory memory of a controller, such as the smart phone of the user. The pre-programmed algorithms of the “app” that resides within the microprocessor apply 609 algorithmic steps to the sensed or registered physical parameter values. As parameter values continue to be dynamically inputted by the sensor, the algorithmic steps continue to provide output feedback 610 to the user, to the user's healthcare provider and/or to the user's EMR. It is to be understood that the specific way that the source code is organized into files, modules and functions is a matter of programmer design choice and is not a limitation of the present invention. It should also be understood, however, that the methodology of the present invention is made possible by virtue of the existence of the internet.

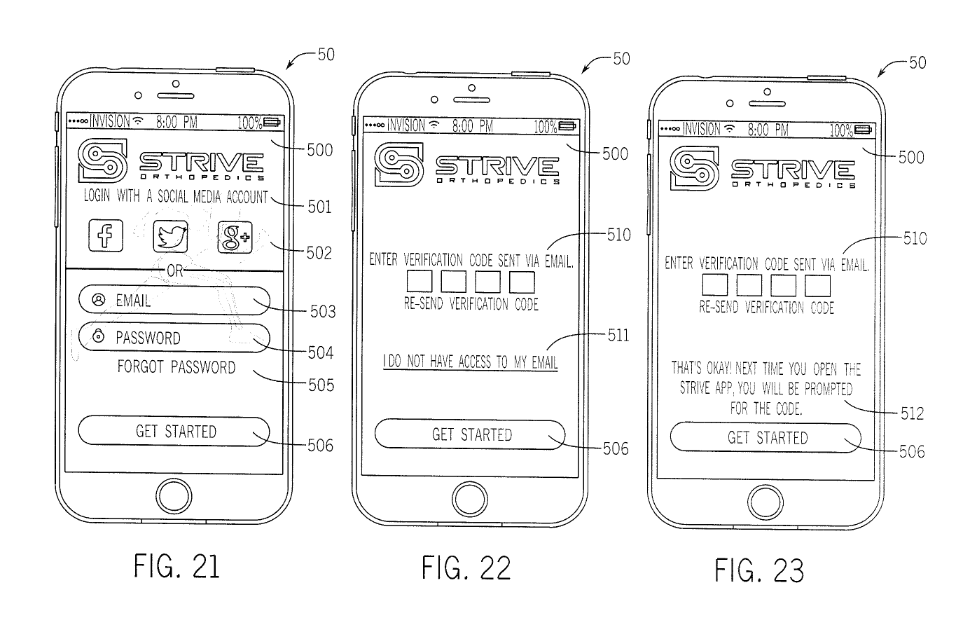

Relative to the “app” that is used in accordance with the present invention, reference is now made to FIGS. 21 through 34, each of which shows a personal computing device 50 having a display screen, generally identified 500. As the user moves through the various iterations of the display screen 500, the content of the display screen 500 will change. Starting with FIG. 21, it shows the display screen 500 that would be shown to the user at the time of initial setup. More specifically, it shows a “log-in” queue 501, which allows the user to log-in via any one or more of the social media accounts 502 shown. Alternatively, the user can log-in using his or her e-mail address 503 together with a password queue 504. If the log-in is validated, the user is prompted to “get started” 506. If, however, the user has forgotten his or her password, the user is queued to retrieve his or her password via a similar prompt 505.

FIG. 22 illustrates another iteration of the screen display 500 through which the user is asked to enter a verification code 510 if he or she has forgotten his or her password. Further, the user may interact with the “app” to inform it that the user does not have access to his or her e-mail 511. As shown in FIG. 23, the user can get started 506 by setting up his or her app via a prompt 512 that will come up the next time the user opens the app. If the user has the correct information, the user is again prompted to get started 506.

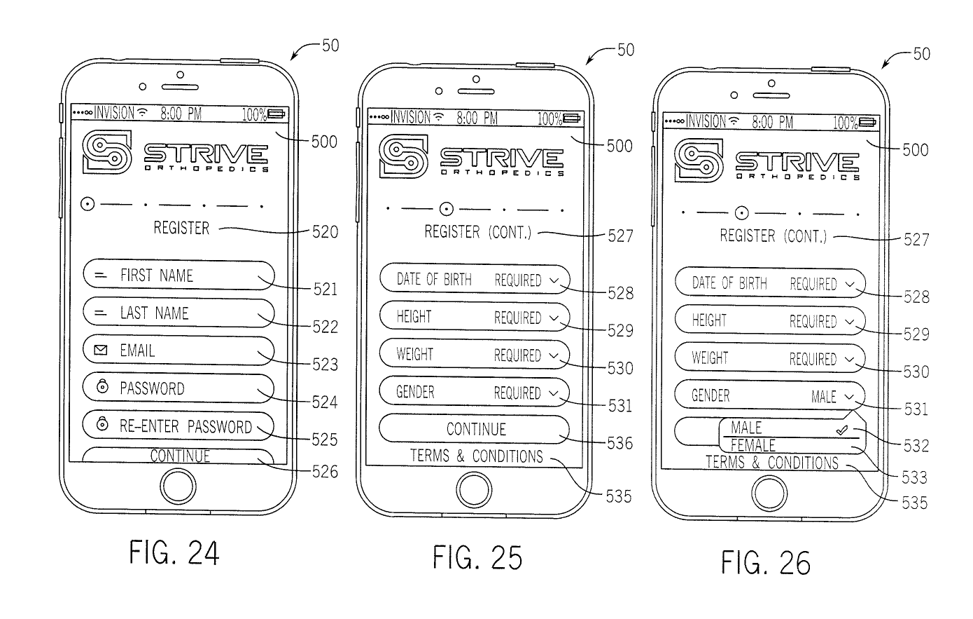

In order for the user to accomplish his or her registration 520 for use of the app, as shown in FIG. 24, the user must enter certain identifying information—his or her first name 521, last name 522, e-mail address 523, password 524, and, for security purposes, a prompt to re-enter the password 525. The user may then continue 526 with the next screen display. Referring now to FIG. 25, it does show that the user has essentially completed one portion of the registration 527. Next, the user is prompted to enter additional identifying information—a date of birth 528, height 529, weight 530, and gender 531. Again, the user is prompted to continue 536 the registration process. Further, the screen display 500 provides user access to the terms of use and conditions 535 for using the app. Referring to FIG. 26, the elements or prompts shown in FIG. 25 are simply repeated with the exception of the user being able to identify his or her gender as male 532 or female 533 via a drop down box from the gender queue 531.

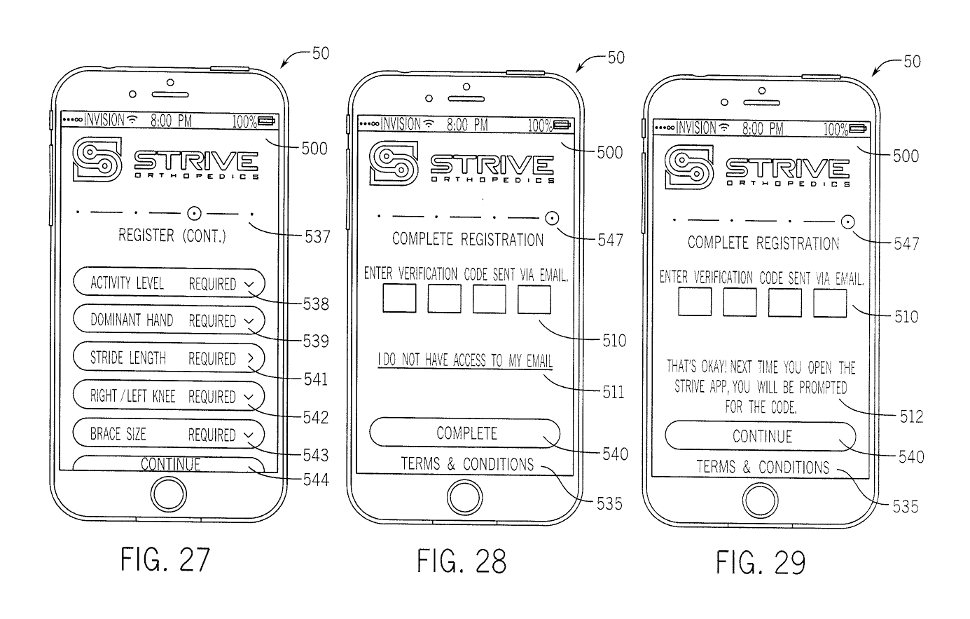

Referring now to FIG. 27, it shows that the user has completed another part of the registration 537. From this screen 500, the user is prompted to enter an activity level 538, his or her dominant hand 539, stride length 541, right or left knee option 542 (although it is to be assumed that other joints could likewise be included in this app which is highly variable in its usage—this example being only one of many different or alternative configurations). The user is also prompted to enter a brace size 543, after which the user can continue 544 with the registration process. Referring to FIG. 28, it shows that the user has completed registration 547 and is then prompted to enter a verification code that is sent to the user via e-mail 510. If the user does not have access to his or her e-mail 511, he or she will be prompted at a later time to enter the verification code 510. The user is then provided with a queue 540 to complete registration. FIG. 29 informs the user that the app will prompt 512 the user to enter the code the next time he or she opens the app. It also informs the user that the registration process has been completed 547.

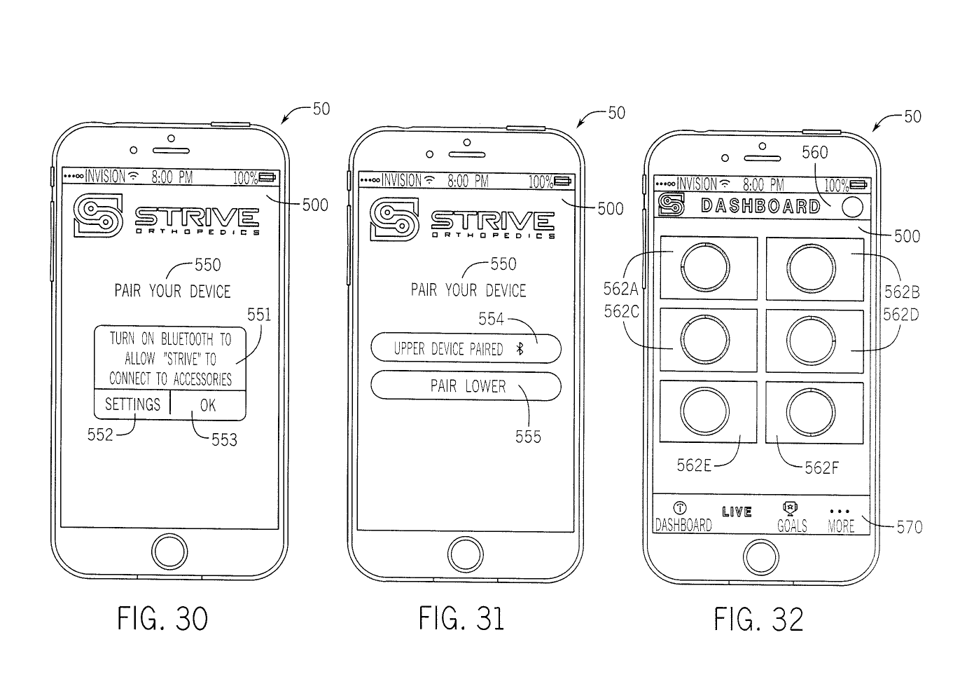

Referring to FIG. 30, it includes a prompt for the user to “pair” his or her device 550, which includes turning on his or her BlueTooth® connectivity 551 and changing settings if required and otherwise activating that connectivity 553. FIG. 31 requests that the user pair 550 his or her upper device 554 and to do the same with the lower device 555. Once the connectivity has been established, a dashboard 560, as is shown in FIG. 32, illustrates a plurality of options for measuring any number of physical parameters or body metrics that are being assessed. These body metrics, shown in the abstract as circles 562A, 562B, 562C, 562D, 562E, 562F, are of any type that has been previously discussed in this detailed description and is to be understood to include any variety of physical measurements, including, without limitation, amounts, amplitudes, displacement, magnitudes, movement, quantifications, ranges of motion, valuations and other desired or required measurements.

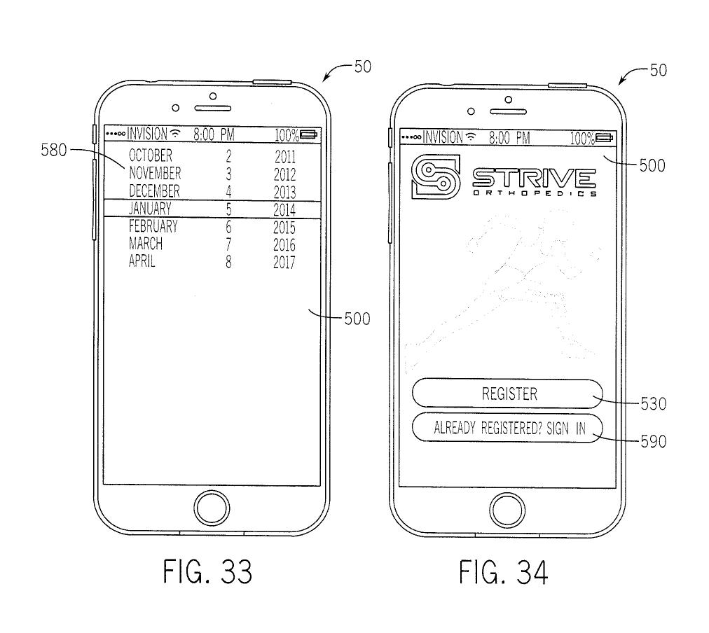

The display 500 as shown in FIG. 32 is at the heart of the “app” that is used in accordance with the present invention. That is, the sensors 32, 34, 42, 44, 402, 412 comprise devices for detecting a wide variety of objectively different physical parameters, such as the amount of light as detected by a light sensor; heat and cold as detected by temperature sensors; movement as detected by motion sensors, applied force as detected by pressure sensors; the presence or absence of certain harmful agents as detected by chemical sensors; electric field sensors; magnetic field sensors; displacement sensors; and acceleration sensors. Sensors of this nature are used in the present application for detecting absolute parameter values and, more importantly, used for detecting parameter deltas. Most importantly, however, is the fact that the “resolution” of a sensor is the smallest change that the sensor can detect in the relevant “quantity” that it is measuring, i.e. temperature measured in tenths of degrees Fahrenheit or Celsius; motion and displacement measured in inches, fractions of inches, millimeters, micrometers and smaller displacement distances; pressure in terms of force per unit area; and so on. In short, while such sensors typically measure “absolutes,” coupled with suitable software, algorithmic steps and memory, the changes in parameters can be detected and monitored as well. These changes, or deltas, are an essential element of the present invention. The dashboard and other prompts are provided at the bottom 570 of the screen display 500. Lastly, FIGS. 33 and 34 provide the user with a visual queue 580 for entering a date and again asking the user to register 530, if he or she has not already done so. If the user has already registered, the user can simply sign in 590 to access the app from the display 500 and proceed to initiation of the app and its preprogrammed sequencing in accordance with that shown in FIG. 20.

Finally, the power supply components 405, 415, 26B of each wearable sensing device provide power required for electromechanical functionality of the various components for the mobile terminal. The power may be provided internally, externally or a combination thereof. In the preferred embodiment, lithium ion batteries 26B, 405, 415 of the type that can recharge via induction charging are provided. However, the batteries could also be replaceable, or even rechargeable via other common “plug-in” charging technology.

In accordance with the foregoing, it will be apparent that a variety of device constructs are contemplated and devised in the wearable sensing devices of the present invention, all of which facilitate the monitoring of physical and physiological parameters in human patients. Such wearable sensing devices are incorporated into a wearable as previously defined, incorporates a small portable power supply; monitoring electronics mounted to or used in the wearable; a processing unit or component; and a memory unit or component to continuously or intermittently record parameter data—such data then being stored in an onboard portable memory unit and/or wirelessly transmitted to another electronic device or devices. In the case of the latter, the wearable would necessarily incorporate a wireless data transmission unit or component. The memory unit could also be synchronized with the processing unit to save and then later download monitoring data for detection of any physical or physiological condition that is benign or a condition that requires mediation of some sort. In all cases, the monitored parameters could be used to inform the patient and his or her healthcare providers of any changes that would require mediation of a medically-problematic condition.

The details of the invention having been disclosed in accordance with the foregoing, I claim:

1. A wearable for the noninvasive medical monitoring of physical and physiological parameters in a human limb of a user, the limb comprising at least one joint and skin that overlays the limb and joint, the wearable comprising:
a pair of wearable sensing devices;
means for positioning each wearable directly or indirectly atop the user's skin, the position of each wearable sensing device being fixed;
a ten axis sensing element integrated into each wearable sensing device;
means for calibrating the integrated ten axis sensing elements in the pair of wearable sensing devices;
means for detecting body metrics via the sensing elements in each wearable sensing device;
means for measuring body metrics via the sensing elements in each wearable sensing device;
a memory for storing the detected and measured body metrics; and
a microprocessor for applying algorithmic steps in accordance with applied quaternion matrix mathematics analysis to assess physical and physiological delta information relative to the user.

2. The wearable according to claim 1, wherein one wearable sensing device is disposed to one side of a body joint and one wearable sensing device is disposed to the other side of the body joint.

3. The wearable according to claim 1, wherein the wearable sensing devices are electronically connected together via an input/output communications wire and wherein at least one of the wearable sensing devices comprises a local low energy wireless transceiver to provide a wireless personal area network for the wearable sensing devices.

4. The wearable according to claim 1 wherein each of the wearable sensing devices comprises its own local low energy wireless transceiver to provide a wireless personal area network for each of the wearable sensing devices.

5. The wearable according to claim 1 further comprising:
a stand-alone processing unit, the processing unit comprising a local low energy wireless transceiver to provide a wireless personal area network for the unit and the sensing devices connected to it; and
an input/output communications wire disposed between each of the wearable sensing devices and the stand-alone processing unit.

6. The wearable according to claim 5 further comprising:
a stretch sensor;
a circumferential band having a fixed length and two ends, the band encircling one part of the user's limb; and
an input/output communications wire disposed between the stretch sensor and the stand-alone processing unit;
wherein the two ends of the circumferential band are used in conjunction with the stretch sensor to detect an increase or a decrease in the circumference of the user's limb.

7. The wearable according to claim 5 further comprising at least one from a group consisting of:
an EMG sensing element and an input/output communications wire disposed between the EMG sensing element and the stand-alone processing unit;
a skin temperature sensing element and an input/output communications wire disposed between the skin temperature sensing element and the stand-alone processing unit; and
at least one skin color sensing element and an input/output communications wire disposed between the skin color sensing element and the stand-alone processing unit.

8. The wearable according to claim 5 further comprising at least one from a group consisting of:
clothing;
a sleeve;
a legging;
a wrap;
a brace;
a support; and
body-attachable patches;
wherein the wearable is comprised of natural fibers, synthetic fibers, plastic materials, metals or a combination thereof.

9. The wearable according to claim 1 further comprising pockets, one pocket for each wearable sensing device and each pocket retaining a wearable sensing device within it and wherein the wearable sensing devices are configured of MEMs circuitry encased within a housing, the housing comprising a tapered nose portion, which nose portion provides the leading edge for the wearable sensing device when inserted into the pocket.

10. The wearable according to claim 1 wherein each wearable sensing device alternatively comprises an integrated ten axis, a nine axis, a six axis or a three axis sensing element and wherein each wearable sensing device comprising a ten axis, a nine axis, a six axis or a three axis sensing element can be combined with another wearable sensing device comprising a ten axis, a nine axis, a six axis or a three axis sensing element.

11. A system for the noninvasive medical monitoring of physical and physiological parameters in a human limb of a user, the limb comprising at least one joint and skin that overlays the limb and joint, the system comprising:
a wearable;
a pair of wearable sensing devices incorporated into the wearable;
means for positioning each wearable directly or indirectly atop the user's skin, the position of each wearable sensing device being fixed;
a ten axis sensing element integrated into each wearable sensing device;
means for calibrating the integrated ten axis sensing elements in the pair of wearable sensing devices;
means for detecting body metrics via the sensing elements in each wearable sensing device;
means for measuring body metrics via the sensing elements in each wearable sensing device;
a memory for storing the detected and measured body metrics;
a microprocessor for applying algorithmic steps in accordance with applied quaternion matrix mathematics analysis to assess physical and physiological delta information relative to the user; and
a portable computing device.

12. The system according to claim 11, wherein the wearable sensing devices are electronically connected together via an input/output communications wire and wherein at least one of the wearable sensing devices comprises a local low energy wireless transceiver to provide a wireless personal area network for the wearable sensing devices such that the network includes the portable computing device.

13. The system according to claim 11 wherein each of the wearable sensing devices comprises its own local low energy wireless transceiver to provide a wireless personal area network for each of the wearable sensing devices such that the network includes the portable computing device.

14. The system according to claim 11 further comprising:
a stand-alone processing unit, the processing unit comprising a local low energy wireless transceiver to provide a wireless personal area network for the unit, the sensing devices connected to it and the portable computing device that is wirelessly connected to the processing unit; and
an input/output communications wire disposed between each of the wearable sensing devices and the stand-alone processing unit.

15. The system according to claim 14 further comprising:
a stretch sensor;
a circumferential band having a fixed length and two ends, the band encircling one part of the user's limb; and
an input/output communications wire disposed between the stretch sensor and the stand-alone processing unit;
wherein the two ends of the circumferential band are used in conjunction with the stretch sensor to detect an increase or a decrease in the circumference of the user's limb.

16. The system according to claim 14 further comprising at least one from a group consisting of:
an EMG sensing element and an input/output communications wire disposed between the EMG sensing element and the stand-alone processing unit;
a skin temperature sensing element and an input/output communications wire disposed between the skin temperature sensing element and the stand-alone processing unit; and
a skin color sensing element and an input/output communications wire disposed between the skin color sensing element and the stand-alone processing unit.

17. The system according to claim 14 further comprising at least one from a group consisting of:
clothing;
a sleeve;
a legging;
a wrap;
a brace;
a support; and
body-attachable patches;
wherein the wearable is comprised of natural fibers, synthetic fibers, plastic materials, metals or a combination thereof.

18. The system according to claim 14 further comprising a pair of pockets defined in the wearable, each pocket retaining a wearable sensing device in it and wherein the wearable sensing devices are configured of MEMs circuitry encased within a housing, the housing comprising a tapered nose portion, which nose portion provides the leading edge for the wearable sensing device when inserted into the pocket.

19. The system according to claim 15 wherein each wearable sensing device alternatively comprises an integrated ten axis, a nine axis, a six axis or a three axis sensing element and wherein each wearable sensing device comprising a ten axis, a nine axis, a six axis or a three axis sensing element can be combined with another wearable sensing device comprising a ten axis, a nine axis, a six axis or a three axis sensing element.

20. A method for noninvasively monitoring of physical and physiological parameters in a human limb of a user, the limb comprising at least one joint and skin that overlays the limb and joint, the method comprising the steps of:
providing a wearable;
incorporating a pair of wearable sensing devices into the wearable;
positioning each wearable directly or indirectly atop the user's skin, the position of each wearable sensing device being fixed;
integrating a ten axis sensing element into each wearable sensing device;
calibrating the integrated ten axis sensing elements in the pair of wearable sensing devices;
detecting body metrics via the sensing elements in each wearable sensing device;
measuring body metrics via the sensing elements in each wearable sensing device;
providing a memory;
storing the detected and measured body metrics in the memory;
providing a microprocessor;
using the microprocessor to apply algorithmic steps in accordance with applied quaternion matrix mathematics analysis to assess physical and physiological delta information relative to the user; and
providing a portable computing device.

21. The method of claim 20 further comprising the steps of electrically connecting the wearable sensing devices and providing a local low energy wireless transceiver within one of the wearable sensing devices to provide a wireless personal area network for the wearable sensing devices such that the network includes the portable computing device.

22. The method of claim 20 further comprising the steps of providing each wearable sensing device with its own local low energy wireless transceiver to provide a wireless personal area network for each of the wearable sensing devices such that the network includes the portable computing device.

23. The method of claim 20 further comprising the steps of:
providing a stand-alone processing unit, the processing unit comprising a local low energy wireless transceiver to provide a wireless personal area network for the unit, the sensing devices connected to it and the portable computing device that is wirelessly connected to the processing unit; and
providing an input/output communications wire between each of the wearable sensing devices and the stand-alone processing unit.

24. The method of claim 23 further comprising the steps of:
providing a stretch sensor;
providing a circumferential band having a fixed length and two ends, the band encircling one part of the user's limb;
providing an input/output communications wire between the stretch sensor and the stand-alone processing unit; and
using the two ends of the circumferential band in conjunction with the stretch sensor to detect an increase or a decrease in the circumference of the user's limb.

25. The method of claim 23 further comprising at least one of the steps from a group consisting of:
providing an EMG sensing element and providing an input/output communications wire between the EMG sensing element and the stand-alone processing unit;
providing a skin temperature sensing element and providing an input/output communications wire between the skin temperature sensing element and the stand-alone processing unit; and
providing at least one skin color sensing element; and
providing an input/output communications wire between the skin color sensing element and the stand-alone processing unit.

26. The method of claim 20 further comprising the step of configuring a wearable from at least one from a group consisting of:
clothing;
a sleeve;
a legging;
a wrap;
a brace;
a support; and
body-attachable patches.

27. The method of claim 26 wherein the wearable is comprised of natural fibers, synthetic fibers, plastic materials, metals or a combination thereof.

28. The method of claim 27 further comprising a pair of pockets defined in the wearable, each pocket retaining a wearable sensing device in it.

29. The method of claim 28 wherein the wearable sensing devices are configured of MEMs circuitry encased within a housing, the housing comprising a tapered nose portion, which nose portion provides the leading edge for the wearable sensing device when inserted into a pocket.

30. The method of claim 20 wherein the sensing element integration step alternatively comprises the step of integrating a ten axis, a nine axis, a six axis or a three axis sensing element into the sensing devices and the step of combining a sensing device comprising a ten axis, a nine axis, a six axis or a three axis sensing element with another wearable sensing device comprising a ten axis, a nine axis, a six axis or a three axis sensing element.
